(19) United States
(12) Patent Application Publication
(10) Pub. No.: US 2007/0106059 A1
(43) Pub. Date: May 10, 2007

(54) Macromolecular antioxidants and polymeric macromolecular antioxidants

(76) Inventors: Ashok L. Cholli, Chelmsford, MA (US); Rajesh Kumar, Dracut, MA (US); Ashish Dhawan, Lowell, MA (US); Suizhou Yang, Lowell, MA (US); Vijayendra Kumar, Dracut, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/588,824

(22) Filed: Oct. 27, 2006

Related U.S. Application Data
(60) Provisional application No. 60/731,125, filed on Oct. 27, 2005.

(51) Int. Cl. C08G 73/06; C07D 251/48; C12P 17/00

(52) U.S. Cl. 528/422; 544/208; 528/423; 435/117

(57) ABSTRACT

Disclosed are macromolecular antioxidants represented by a structural formula selected from I-VI: 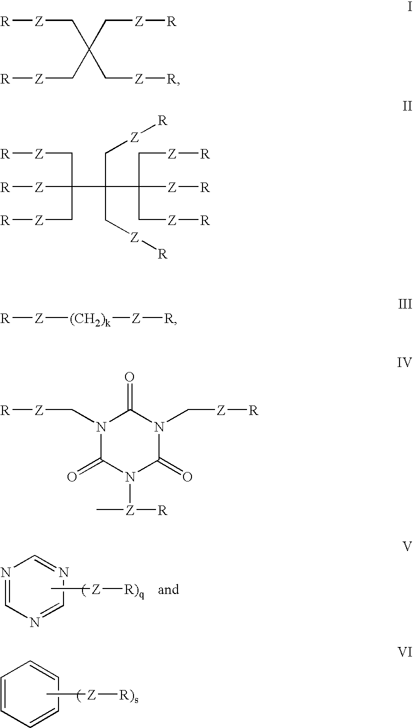
and polymeric macromolecular antioxidants comprises at least one repeating unit represented by a structural formula selected from VIIa, VIIb, VIIIa, VIIIb or a combination thereof: 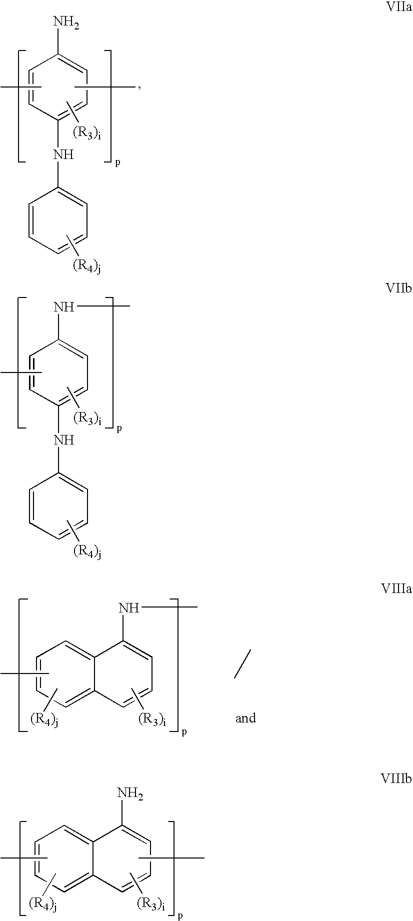 possessing superior oxidative resistance and higher thermal stability than commercially available antioxidants, and synthesis and applications of these macromolecular antioxidants and polymeric macromolecular antioxidants.

### RELATED APPLICATION(S)

This application claims the benefit of U.S. Provisional Application No. 60/731,125 filed on Oct. 27, 2005. The entire teachings of the above application(s) are incorporated herein by reference.

### BACKGROUND OF THE INVENTION

Antioxidants are employed to prevent oxidation in a wide range of materials, for example, plastics, elastomers, lubricants, petroleum based products (lubricants, gasoline, aviation fuels, and engine oils), cooking oil, cosmetics, processed food products, and the like. While many small molecule antioxidants exist, there is a continuing need for new antioxidants that have improved properties.

### SUMMARY OF THE INVENTION

In a particular embodiment, the present invention pertains to macromolecular antioxidants and polymeric macromolecular antioxidants possessing superior oxidative resistance and higher thermal stability than commercially available antioxidants.

In certain particular embodiments, the present invention pertains to macromolecular antioxidants represented by a structural formula selected from I-VI:

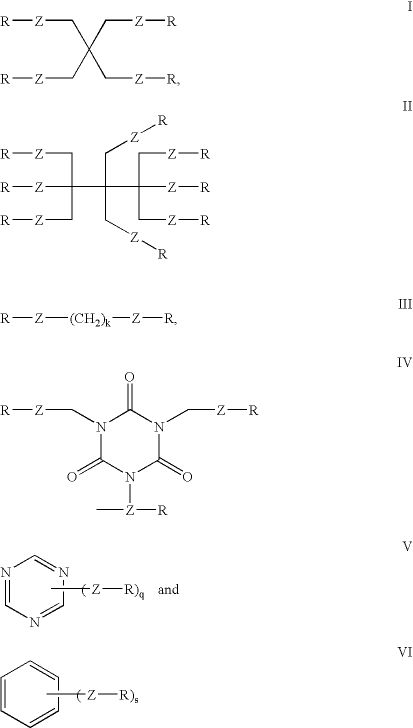

wherein:

Z in each occurrence, independently is a bond, an optionally substituted alkylene group, —S—, —O— or —NH—;

k is a positive integer from 1 to 12;

q is a positive integer from 1 to 3;

s is a positive integer from 1 to 6;

R is:

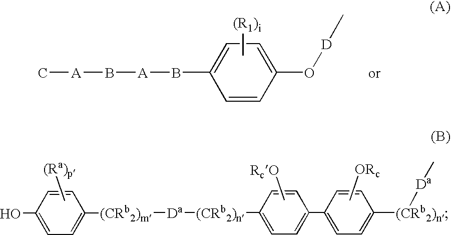

wherein:
A in each occurrence, independently is a bond, —O—, —NH—, —S—, —C(O)—, —C(O)NH—, —NHC(O)—, —C(O)O—, —OC(O)—, —CH═N— or —N═CH—;
B in each occurrence, independently is a bond or an optionally substituted alkyl group;
C in each occurrence independently is -H, an optionally substituted alkyl group or 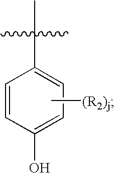
R₁ and R₂ in each occurrence, independently is an optionally substituted alkyl, optionally substituted aryl or optionally substituted aralkyl;
i and j in each occurrence, independently is 0, 1, 2, 3 or 4;
D in each occurrence, independently is a bond, an optionally substituted alkyl group, —(CH₂)₁C(O)O(CH₂)ₕ—, —CH₂)₁NHC(O)(CH₂)ₕ—, —(CH₂)₁C(O)NH(CH₂)ₕ—, —(CH₂)₁OC(O)(CH₂)ₕ—, —(CH₂)₁CH═N(CH₂)ₕ—, —(CH₂)₁N═CH(CH₂)ₕ—, —(CH₂)₁NH(CH₂)ₕ—, —(CH₂)₁S—(CH₂)ₕ—, —(CH₂)₁O(CH₂)ₕ—, —(CH₂)₁C(O)(CH₂)ₕ—,
1 is 0 or a positive integer from 1 to 12;
h is 0 or a positive integer from 1 to 12;
D^{a}, for each occurrence, is independently —C(O)NR_{d}—, —NR_{d}C(O)—, —NR_{d}—, —CR_{d}═N—, —C(O)—, —C(O)O—, —OC(O)—, —O—, —S—, —C(O)OC(O)— or a bond, wherein R_{d} is independently H or optionally substituted alkyl;
R_{c} and R_{c}′ are independently H or an optionally substituted alkyl;
R^{a}, for each occurrence, is independently an optionally substituted alkyl, optionally substituted aryl, optionally substituted alkoxycarbonyl, optionally substituted ester, —OH, —NH₂, —SH;
R^{b}, for each occurrence, is independently H or optionally substituted alkyl;
p′, for each occurrence, is independently an integer from 0 to 4; and
m′ and n′, for each occurrence, are independently integers from 0 to 6.

In certain other particular embodiments the present invention pertains to polymeric macromolecular antioxidants comprising at least one repeating unit represented by a structural formula selected from VIIa, VIIb, VIIIa, VIIIb or a combination thereof: 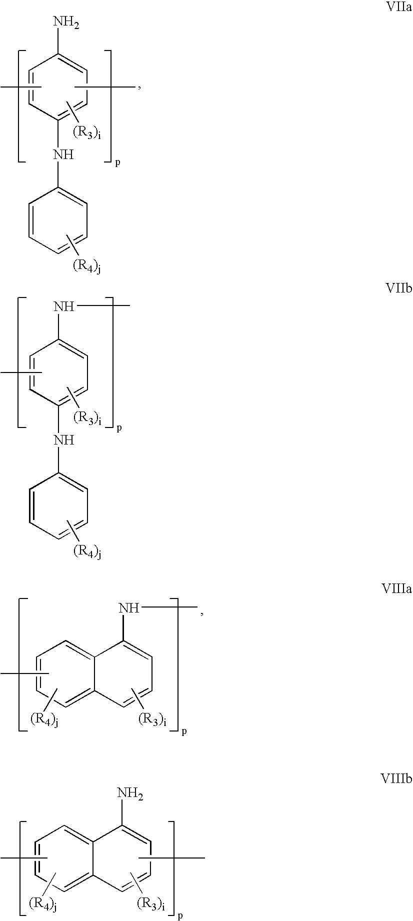
wherein:

R₃ and R₄ in each occurrence, independently is C1-C16 alkyl, —O—C1-C16 alkyl,

—NHAr, —NH₂, —OH, or —SH;

i and j in each occurrence, independently is 0, 1, 2, 3 or 4; and

p in each occurrence, independently is an integer equal to or greater than 2.

In another embodiment, the present invention pertains to methods of preventing oxidation; The method comprises combining an oxidizable material with a compound or polymer of the present invention.

In yet another embodiment, the present invention pertains to methods for preparing compounds represented by a structural formula selected from I-VI. The method comprises comprising the step of reacting R⁺⁺, wherein R⁺⁺ is: 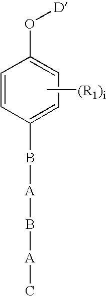
with a compound selected from:

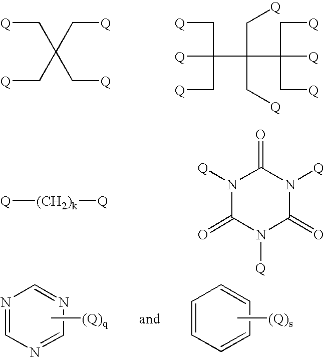

Q is a halogen or -Z-H.

D′ in each occurrence, independently is —H, an optionally substituted alkyl group, —(CH₂)₁C(O)O(CH₂)ₕR*—, —CH₂)₁NHC(O)(CH₂)ₕR*—, —(CH₂)₁C(O)NH(CH₂)ₕR*—, —(CH₂)₁C(O)O(CH₂)ₕR*—, —(CH₂)₁OC(O)(CH₂)ₕR*—, —(CH₂)₁CH═N(CH₂)ₕR*—, —(CH₂)₁N═CH(CH₂)ₕR*—, —(CH₂)₁NH(CH₂)ₕR*—, —(CH₂)₁S—(CH₂)ₕR*—, —(CH₂)₁O(CH₂)ₕR*— or —(CH₂)₁C(O)(CH₂)ₕR*—.

R* in each occurrence, independently is —CH₃ or —H.

In yet another embodiment, the present invention pertains to methods for preparing polymers represented by a structural formula selected from VII and VIII. The method comprises comprising the step of polymerizing a monomer represented by a structural formula selected from: 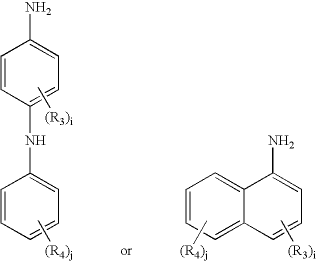
or combinations thereof in the presence of an oxidative polymerization catalyst.

In yet another embodiment the present invention pertains to the use of the disclosed compounds and polymers as antioxidants in a wide range of materials including, but not limited to, food, plastics, elastomers, composites and petroleum based products.

The macromolecular antioxidants and polymeric macromolecular antioxidants of the present invention generally can be synthesized more cost effectively than currently available antioxidants. Macromolecular antioxidants of the present invention can impart high antioxidant activities along with improved thermal stability and performance to a wide range of materials, including but not limited to plastics, elastomers, lubricants, petroleum based products (lubricants, gasoline, aviation fuels, and engine oils), cooking oil, cosmetics, processed food product, than commercially available antioxidants. The macromolecular antioxidants of the present invention generally have higher thermal stability, higher oxidative induction time lower changes in melt flow and diffusion rate than commercially available antioxidants.

### BRIEF DESCRIPTION OF THE DRAWINGS

FIG. 1, is an infrared (IR) spectrum of 1,6-bis[N-(4-hydroxyphenyl)-3-(2,6-di-tert-butyl, 4-hydroxyphenyl)propionamide]hexyl ether of the invention.

FIG. 2 is an ultraviolet (UV) spectrum of 1,6-bis[N-(4-hydroxyphenyl)-3-(2,6-di-tert-butyl, 4-hydroxyphenyl)propionamide]hexyl ether of the invention.

FIG. 3 is a comparison of an oxidative induction time (OIT) of one embodiment of the invention, namely, 1,6-bis[N-(4-hydroxyphenyl)-3-(2,6-di-tert-butyl, 4-hydroxyphenyl)propionamide]hexyl ether, versus commercially available Irganox®.

FIG. 4 is a thermogravimetric analysis (TGA) of 1,6-bis[N-(4-hydroxyphenyl)-3-(2,6-di-tert-butyl, 4-hydroxyphenyl)propionamide]hexyl ether of the invention.

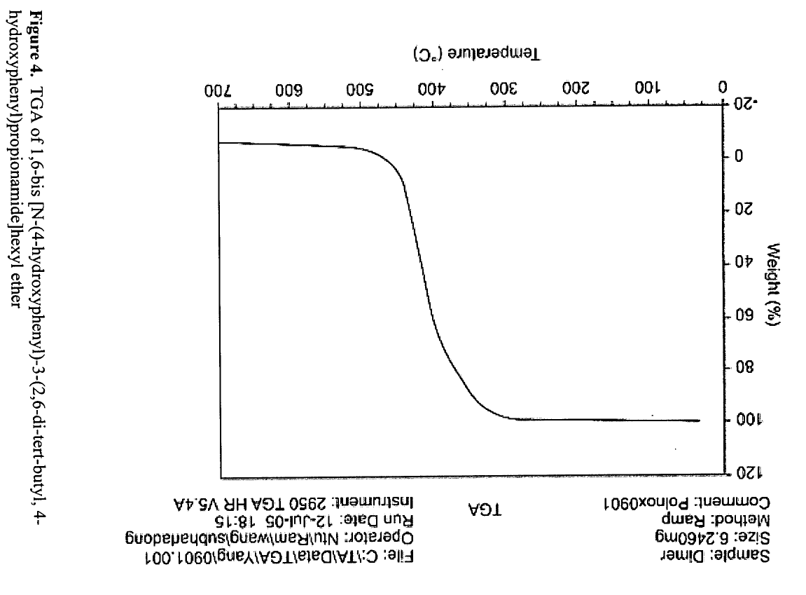

FIG. 5 is an oxidative induction time (OIT) of polypropylene in combination with one embodiment of the invention, namely, 1,6-bis[N-(4-hydroxyphenyl)-3-(2,6-di-tert-butyl, 4-hydroxyphenyl)propionamide]hexyl ether.

FIG. 6 is an oxidative induction time (OIT) of polyol ester based samples in combination with various polymeric macromolecular antioxidants of the present invention versus commercially used APAN (alkylated phenyl naphthalene amine) and DODP (di-octylated diphenyl amine).

FIG. 7 is an oxidative induction time (OIT) for polypropylene in combination with N-phenyl-para-phenylene-diamine versus polypropylene in combination with commercially available Irganox®.

### DETAILED DESCRIPTION OF THE INVENTION

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention. Unless otherwise stated, substituents identified represent options that can occur independently of other listed optional substituents in each occurrence.

In certain embodiments, the present invention pertains to macromolecular antioxidants represented by a structural formula selected from:

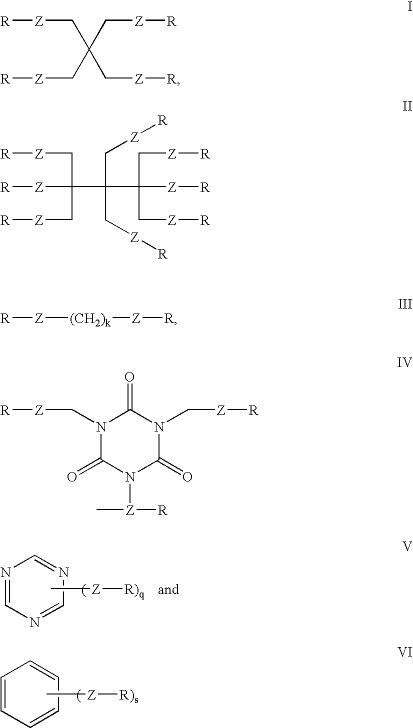

R is:

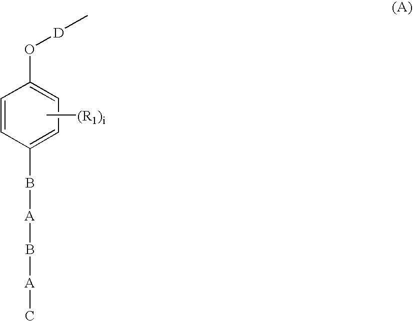

A in each occurrence, independently is a bond, —O—, —NH—, —S—, —C(O)—, —C(O)NH—, —NHC(O)—, —C(O)O—, —OC(O)—, —CH═N— or —N═CH—. In certain particular embodiments, A in each occurrence, independently is —C(O)NH— or —NHC(O)—.

B in each occurrence, independently is a bond or an optionally substituted alkylene group. In certain particular embodiments B is a C1-C6 alkyl.

C in each occurrence, independently is —H, an optionally substituted alkyl group or

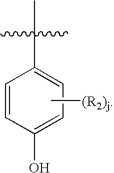

In a particular embodiment, C is:

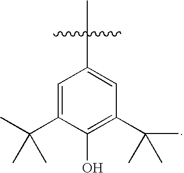

In a particular embodiment R is:

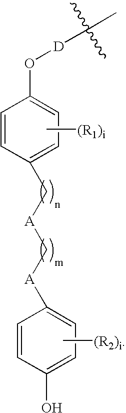

In another particular embodiment R is:

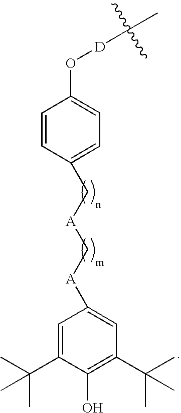

In yet another particular embodiment R is:

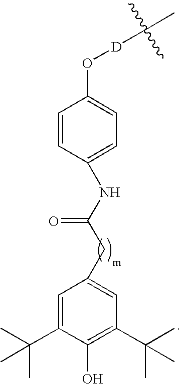

R₁ and R₂ in each occurrence, independently is an optionally substituted alkyl, optionally substituted aryl or optionally substituted aralkyl. In one embodiment, each R₁ and R₂ in each occurrence, independently is an optionally substituted alkyl. In another embodiment, each R₁ and R₂ in each occurrence, independently is a C1-C6 alkyl.

D in each occurrence, independently is a bond, an optionally substituted alkylene group, —(CH₂)₁C(O)O(CH₂)ₕ—, —(CH₂)₁NHC(O)(CH₂)ₕ—, —(CH₂)₁C(O)NH(CH₂)ₕ—, —(CH₂)₁C(O)O(CH₂)ₕ—, —(CH₂)₁OC(O)(CH₂)ₕ—, —(CH₂)₁CH═N(CH₂)ₕ—, —(CH₂)₁N═CH(CH₂)ₕ—, —(CH₂)₁NH(CH₂)ₕ—, —(CH₂)₁S—(CH₂)ₕ—, —(CH₂)₁O(CH₂)ₕ— or —(CH₂)₁C(O)(CH₂)ₕ—.

Z in each occurrence, independently is a bond, an optionally substituted alkylene group, —S—, —O— or —NH—. In a particular embodiment, Z is a single bond.

i and j in each occurrence, independently is 0, 1, 2, 3 or 4. In one embodiment i and j in each occurrence, independently is 0, 1 or 2. In a particular embodiment, i is 0. In another particular embodiment, j is 2.

k is a positive integer from 1 to 20. In one embodiment, k is a positive integer from 1 to 12. In another embodiment, k is a positive integer from 1 to 6.

1 is 0 or a positive integer from 1 to 20, and when D is —(CH₂)₁NHC(O)(CH₂)ₕ—, —(CH₂)₁OC(O)(CH₂)ₕ—, —(CH₂)₁S—(CH₂)ₕ—, or —(CH₂)₁O(CH₂)ₕ₋, 1 is not 0. In one embodiment, 1 is 0 or a positive integer from 1 to 12. In another embodiment, 1 is 0 or a positive integer from 1 to 6.

h is 0 or a positive integer from 1 to 20, When Z is not a bond and D is —(CH₂)₁C(O)O(CH₂)ₕ—, —(CH₂)₁C(O)NH(CH₂)ₕ—, —(CH₂)₁C(O)O(CH₂)ₕ—, —(CH₂)₁NH(CH₂)ₕ—, —(CH₂)₁S—(CH₂)ₕ—, or —(CH₂)₁O(CH₂)ₕ—, h is not 0. In one embodiment, h is 0 or a positive integer from 1 to 12. In another embodiment, h is 0 or a positive integer from 1 to 6. In another embodiment, h is 0.

n and m in each occurrence independently is 0 or a positive integer. In one embodiment, n and m in each occurrence independently is 0 to 18. In another embodiment, n and m in each occurrence independently is 0 to 12. In yet another embodiment, n and m are in each occurrence independently is 0 to 6.

s is a positive integer from 1 to 6.

q is a positive integer from 1 to 3.

In certain embodiments, the present invention is directed to macromolecular antioxidants represented by structural formula I.

In certain embodiments, the present invention is directed to macromolecular antioxidants represented by structural formula II.

In certain embodiments, the present invention is directed to macromolecular antioxidants represented by structural formula III.

In certain embodiments, the present invention is directed to macromolecular antioxidants represented by structural formula IV.

In certain embodiments, the present invention is directed to macromolecular antioxidants represented by structural formula V.

In certain embodiments, the present invention is directed to macromolecular antioxidants represented by structural formula VI.

In other certain embodiments, the present invention is directed to macromolecular antioxidants represented by a structural formula selected from Structural Formulas I-VI, wherein R is:

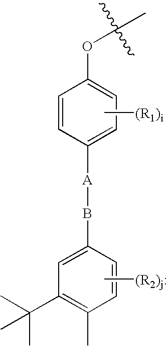

R₁ and R₂ in each occurrence, independently is —H, —OH, a C1-C10 alkyl group or a tert-butyl group; A is —NHC(O)— or —C(O)O— and B is a bond or a C1-C24 alkylene, and i and j are 0, 1, 2, 3 or 4.

In other certain embodiments, the present invention is directed to macromolecular antioxidants represented by a structural formula selected from Structural Formulas I-VI, wherein R is:

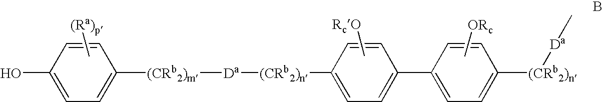

wherein:

D^{a}, for each occurrence, is independently —C(O)NR_{d}—, —NR_{d}C(O)—, —NR_{d}—, —CR_{d}═N—, —C(O)—, —C(O)O—, —OC(O)—, —O—, —S—, —C(O)OC(O)— or a bond. In certain other embodiments D^{a} is —C(O)O—, —OC(O)—, —C(O)NH—, —NHC(O)—, —NH—, —O— or —C(O)—. In certain other embodiments, D^{a} is —NH—, —C(O)NH— or —NHC(O)—. Optionally, D^{a} is not —C(O)O—, —OC(O)—, —O— or —NH—. In various embodiments, the present invention relates to a compound of Structural Formula I and the attendant definitions, wherein D^{a} is —OC(O)—. In another embodiment, D^{a} is —C(O)O—. In another embodiment, D^{a} is —C(O)NH—. In another embodiment, D^{a} is —NHC(O)—. In another embodiment, D^{a} is —NH—. In another embodiment, D^{a} is —CH═N—. In another embodiment, D^{a} is —C(O)—. In another embodiment, D^{a} is —O—. In another embodiment, D^{a} is —C(O)OC(O)—. In another embodiment, D^{a} is a bond.

Each R_{d} is independently —H or optionally substituted alkyl. In certain other embodiments R_{d} is —H or an alkyl group. In certain other embodiments R_{d} is —H or a C1-C10 alkyl group. In certain other embodiments R_{d} is —H.

R_{c} and R_{c}′ are independently H or an optionally substituted alkyl. In one embodiment, R_{c} and R_{c}′ are H. In another embodiment, one of R_{c} and R_{c}′ is H and the other is an optionally substituted alkyl. More specifically, the alkyl is a C1-C10 alkyl. Even more specifically, the alkyl is a C10 alkyl.

R^{a}, for each occurrence, is independently an optionally substituted alkyl, optionally substituted aryl, optionally substituted alkoxycarbonyl, optionally substituted ester, —OH, —NH₂, or —SH. In certain other embodiments, each R^{a} is independently an optionally substituted alkyl or optionally substituted alkoxycarbonyl. In certain other embodiment each R^{a} is independently an alkyl or alkoxycarbonyl. In certain other embodiments each R^{a} is independently a C₁-C₆ alkyl or a C₁-C₆ alkoxycarbonyl. In certain other embodiments each R^{a} is independently tert-butyl or propoxycarbonyl. In certain other embodiments each R^{a} is independently an alkyl group. In certain embodiments each R^{a} is independently a bulky alkyl group. Suitable examples of bulky alkyl groups include butyl, sec-butyl, tert-butyl, 2-propyl, 1,1-dimethylhexyl, and the like. In certain embodiments each R^{a} is tert-butyl. In certain embodiments at least one R^{a} adjacent to the —OH group is a bulky alkyl group (e.g., butyl, sec-butyl, tert-butyl, 2-propyl, 1,1-dimethylhexyl, and the like). In certain other embodiments both R^{a} groups adjacent to —OH are bulky alkyl groups (e.g., butyl, sec-butyl, tert-butyl, 2-propyl, 1,1-dimethylhexyl, and the like). In another embodiment, both R^{a} groups are tert-butyl. In another embodiment, both R^{a} groups are tert-butyl adjacent to the OH group.

R^{b}, for each occurrence, is independently H or optionally substituted alkyl. In certain embodiment, R^{b} is H.

Each n′ and m′ are independently integers from 0 to 18. In another embodiment, n′ and m′ in each occurrence, independently is 0 to 12. In yet another embodiment, n′ and m′ in each occurrence, independently is 0 to 6. In certain embodiments each n′ and m′ are independently integers from 0 to 2. In a specific embodiment, n′ is 0. In another specific embodiment, m is an integer from 0 to 2. In another specific embodiment, n′ is 0 and m′ is 2.

Each p′ is independently an integer from 0 to 4. In certain embodiments, each p′ is independently an integer from 0 to 2. In certain embodiments, p′ is 2.

In an additional embodiment, for formulas I-VI R is:

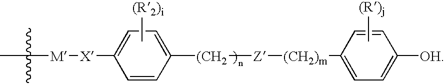

n and m in each occurrence, independently is 0 or a positive integer. In one embodiment, n and m in each occurrence, independently is 0 to 18. In another embodiment, n and m in each occurrence, independently is 0 to 12. In yet another embodiment, n and m in each occurrence, independently is 0 to 6.

i and j in each occurrence, independently is 0, 1, 2, 3 or 4. In one embodiment, i and j in each occurrence, independently is 0, 1 or 2. In a particular embodiment, i is 0. In another particular embodiment, j is 2.

Z′ is —C(O)O—, —OC(O)—, —C(O)NH—, —NHC(O)—, —NH—, —CH═N—, —C(O)—, —O—, —S—, —C(O)OC(O)— or a bond. In one embodiment, Z′ is —C(O)O—. In another embodiment, Z′ is —OC(O)—. In yet another embodiment, Z′ is —C(O)NH—. In yet another embodiment, Z′ is —NHC(O)—. In yet another embodiment, Z′ is —NH—. In yet another embodiment, Z′ is —CH═N—. In yet another embodiment, Z′ is —C(O)—. In yet another embodiment, Z′ is —O—. In yet another embodiment, Z′ is —S—. In yet another embodiment, Z′ is —C(O)OC(O)—. In yet another embodiment, Z′ is a bond.

R′ is an optionally substituted C1-C6 alkyl, —OH, —NH₂, —SH, an optionally substituted aryl, an ester or

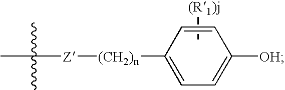

wherein at least one R′ adjacent to the —OH group is an optionally substituted bulky alkyl group (e.g., butyl, sec-butyl, tert-butyl, 2-propyl, 1,1-dimethylhexyl, and the like).

R′₁ is an optionally substituted C1-C6 alkyl, an optionally substituted aryl, an optionally substituted aralkyl, —OH, —NH₂, —SH, or C1-C6 alkyl ester wherein at least one R₁ adjacent to the —OH group is a bulky alkyl group (e.g., butyl, sec-butyl, tert-butyl, 2-propyl, 1,1-dimethylhexyl, and the like).

R′₂ is an optionally substituted C1-C6 alkyl, an optionally substituted aryl, an optionally substituted aralkyl, —OH, —NH₂, —SH, or ester.

X′ is —C(O)O—, —OC(O)—, —C(O)NH—, —NHC(O)—, —NH—, —CH═N—, —C(O)—, —O—, —S—, —C(O)OC(O)— or a bond. In one embodiment X′ is —C(O)O—. In another embodiment X′ is —OC(O)—. In yet another embodiment X′ is —C(O)NH—. In yet another embodiment X′ is —NHC(O)—. In yet another embodiment X′ is —NH—. In yet another embodiment X′ is —CH═N—. In yet another embodiment X′ is —C(O)—. In yet another embodiment X′ is —O—. In yet another embodiment X′ is —S—. In yet another embodiment X′ is —C(O)OC(O)—. In yet another embodiment X′ is a bond.

M′ is H, an optionally substituted aryl, an optionally substituted C1-C20 linear or branched alkyl chain with or without any functional group anywhere in the chain, or

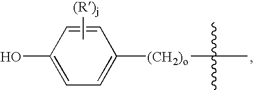

o is 0 or a positive integer. Preferably o is 0 to 18. More preferably o is 0 to 12.

Even more preferably is 0 to 6.

In yet another embodiment, for formulas I-VI R is:

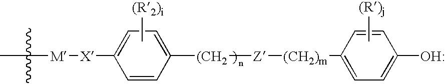

R′₂ is C1-C6 alkyl, —OH, —NH₂, —SH, aryl, ester, aralkyl or

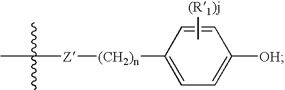

wherein at least one R′₂ is —OH, and the values and preferred values for the remainder of the variables for R are as described immediately above.

In a specific embodiment, M′ is

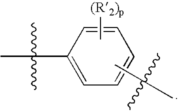

wherein p is 0, 1, 2, 3 or 4; and the values and preferred values for the remainder of the variables are as described above for formulas I-VI.

In an additional embodiment, the present invention is directed to macromolecular antioxidants of formulas I-VI, wherein:

Z is a single bond; and

R is represented by the following structural formula:

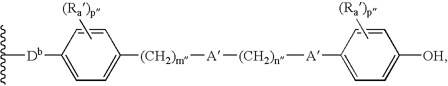

wherein:
D^{b} for each occurrence, is independently —O—, —NH—, —C(O)NH—, —NHC(O)—, —C(O)O—, —OC(O)— and —CH₂—;
Rₐ′ for each occurrence, is independently H, optionally substituted alkyl or optionally substituted aryl;
A′, for each occurrence, is independently —O—, —NH—, —C(O)NH—, —NHC(O)—, —C(O)O—, —OC(O)— and —CH₂—;
m″ and n″ are independently 0 or an integer from 0 to 12; and
p″, for each occurrence, is independently 0, 1, 2, 3 or 4.

Examples of macromolecular antioxidants of the present invention, for example, high molecular weight dimers, and tetramers are shown below.

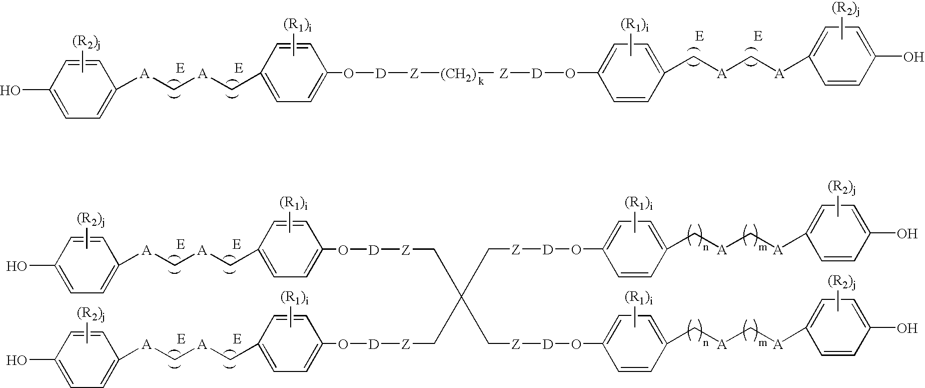

In a first specific embodiment, the present invention is directed to macromolecular antioxidants of Structural Formulas I-VI, wherein R is represented by Structural Formula B,

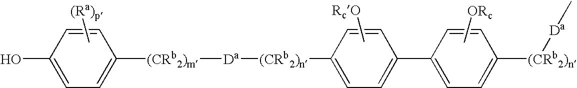

wherein:

Z is a bond;

D^{a}, for each occurrence, is independently —C(O)O—, —OC(O)—, —C(O)NH—, —NHC(O)—, —NH—, —O— or —C(O)—;

R^{b} is H;

R^{a}, for each occurrence is independently an optionally substituted alkyl or

optionally substituted alkoxycarbonyl;

n′ and m′, for each occurrence, are independently integers from 0 to 2;

p′, for each occurrence, is independently an integer from 0 to 2; and the remainder of the variables are as described above for Structural Formula B.

In a second specific embodiment, for macromolecular antioxidants of Structural Formulas I-VI, R is represented by Structural Formula B, wherein:

D^{a}, for each occurrence, is independently —NH—, —C(O)NH— or —NHC(O)—;

R^{a}, for each occurrence is independently an alkyl or an alkoxycarbonyl;

p′ is 2; and the remainder of the variables are as described in the first embodiment.

In a third specific embodiment, for macromolecular antioxidants of Structural Formulas I-VI, R is represented by Structural Formula B, wherein:

Each R^{a} is independently an alkyl group, and the remainder of the variables are as described above in the third embodiment. In certain embodiments each R^{a} is a bulky alkyl group. In certain embodiments two R^{a} groups are bulky alkyl groups adjacent to the —OH group. In certain embodiments the two R groups are tert-butyl groups adjacent to the —OH group.

In a fourth specific embodiment, for macromolecular antioxidants of Structural Formulas I-VI, R is represented by Structural Formula B1, wherein:

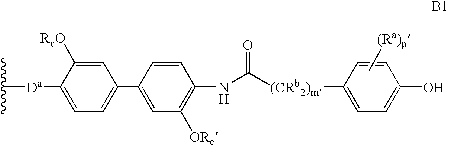

Z is a bond;

D^{a} is —NH—, —C(O)NH— or —NHC(O)—;

R^{a}, for each occurrence, is independently an optionally substituted alkyl, optionally substituted aryl, optionally substituted alkoxycarbonyl, optionally substituted ester, —OH, —NH₂, or —SH;

R^{b}, for each occurrence, is independently H or optionally substituted alkyl.

p′, for each occurrence, is independently an integer from 0 to 4;

m′, for each occurrence, is independently an integer from 0 to 6; and

R_{c} and R_{c}′ are independently H or optionally substituted alkyl and at least one of R_{c} and R_{c}′ is H. In certain embodiments, R_{c} and R_{c}′ are H. In certain other embodiments, one of R_{c} and R_{c}′ is H and the other is an alkyl group. More specifically, the alkyl-group is a C1-C10 alkyl. Even more specifically, the alkyl group is a C10 alkyl.

In a fifth specific embodiment, for macromolecular antioxidants of Structural Formulas I-VI, R is represented by Structural Formula B1, wherein:

R^{a}, for each occurrence, is independently an optionally substituted alkyl;

R^{b} is H;

p′, for each occurrence, is independently an integer from 0 to 2;

m′, for each occurrence, is independently an integer from 0 to 2; and the remainder of the variables are as described above in the fourth embodiment.

In a sixth specific embodiment, for macromolecular antioxidants of Structural Formulas I-VI, R is represented by Structural Formula B1, wherein each R^{a} is independently an alkyl group, and the remainder of the variables are as described above in the sixth embodiment. In certain embodiments each R^{a} is a bulky alkyl group. In certain embodiments two R^{a} groups are bulky alkyl groups adjacent to the —OH group. In certain embodiments the two R groups are tert-butyl groups adjacent to the —OH group.

In a seventh specific embodiment, for macromolecular antioxidants of Structural Formulas I-VI, R is represented by Structural Formula B2, wherein:

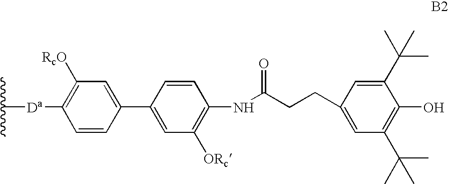

Z is a single bond;

D^{a} is —NH—, —C(O)NH— or —NHC(O)—;

R_{c} and R_{c}′ are independently H or optionally substituted alkyl and at least one of R_{c} and R_{c}′ is H.

In a eighth specific embodiment, for macromolecular antioxidants of Structural Formulas I-VI, R is represented by Structural Formula B2, wherein one of R_{c} and R_{c}′ is H and the other is an alkyl group. More specifically, the alkyl group is a C1-C10 alkyl. Even more specifically, the alkyl group is a C₁₀ alkyl.

In a ninth specific embodiment, for macromolecular antioxidants of Structural Formulas I-VI, R is represented by Structural Formula B3:

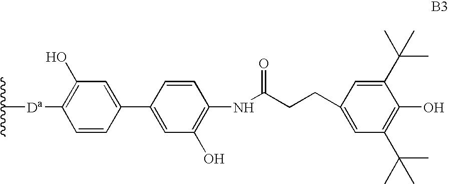

wherein Z is a bond and D^{a} is —NH—, —C(O)NH— or —NHC(O)—.

In a tenth specific embodiment, for macromolecular antioxidants of Structural Formulas I-VI, R is represented by Structural Formula A:

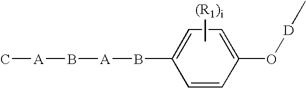

wherein Z is a bond and the remainder of the variables are as described above for Structural Formula A.

In a eleventh specific embodiment, for macromolecular antioxidants of Structural Formulas I-VI, R is represented by Structural Formula A, wherein h is 0 and the remainder of the variables are as described in the tenth specific embodiment.

In a twelfth specific embodiment, for macromolecular antioxidants of Structural Formulas I-VI, R is represented by Structural Formula A1

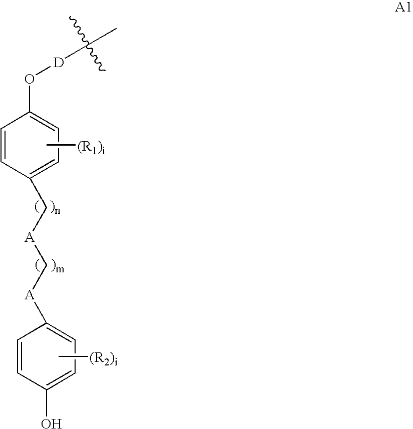

wherein the variables are as described as in the tenth specific embodiment. More specifically, D is —(CH₂)₁—C(O)O—, —(CH₂)₁—C(O)NH— or a bond.

In a thirteenth specific embodiment, for macromolecular antioxidants of Structural Formulas I-VI, R is represented by Structural Formula A2:

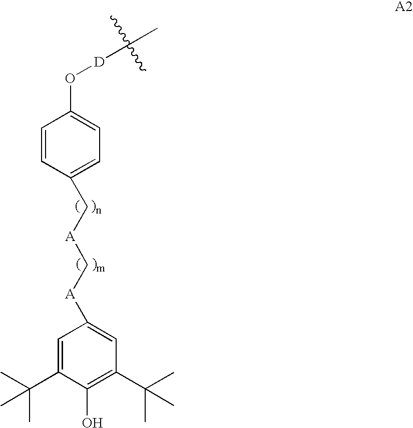

wherein the variables are as described in the tenth specific embodiment. More specifically, D is —(CH₂)₁—C(O)O—, —(CH₂)₁—C(O)NH— or a bond.

In a fourteenth specific embodiment, for macromolecular antioxidants of Structural Formulas I-VI, R is represented by Structural Formula A3

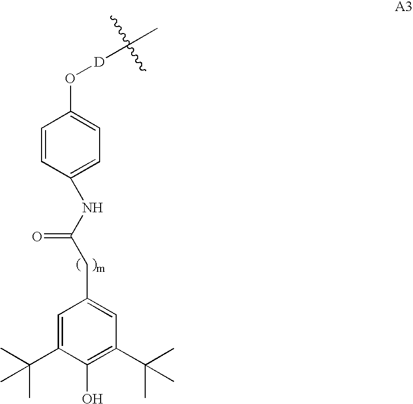

wherein the variables are as described as in the tenth specific embodiment. More specifically, D is —(CH₂)₁—C(O)O—, —(CH₂)₁—C(O)NH— or a bond. Even more specifically, m is 2.

In a fifteenth specific embodiment, for macromolecular antioxidants of Structural Formulas I-VI, R is represented by Structural Formula A4

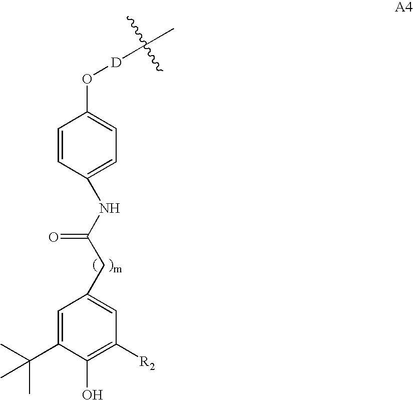

wherein the variables are as described in the tenth specific embodiment. More specifically, D is —(CH₂)₁—C(O)O—, —(CH₂)₁—C(O)NH— or a bond. Even more specifically, m is 2. Even more specifically, m is 2 and R₂ is -Me.

In certain embodiments the present invention pertains to methods of synthesizing compounds represented by a structural formula selected from I-VI, comprising the step of reacting R⁺⁺, wherein R⁺⁺ is 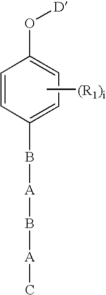
with a compound selected from:

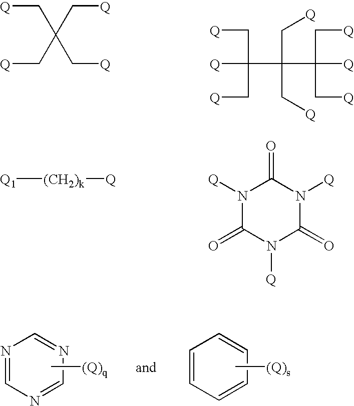

Q is an electrophilic group or a leaving group, such as for example, a halogen, for example, fluorine, chlorine, bromine or iodine, or Q is -Z-H where Z and the remainder of the variables are as described above.

D′ in each occurrence, independently is —H, an optionally substituted alkyl group, —(CH₂)₁C(O)O(CH₂)ₕR*—, —(CH₂)₁NHC(O)(CH₂)ₕR*—, —(CH₂)₁C(O)NH(CH₂)ₕR*—, —(CH₂)₁C(O)O(CH₂)ₕR*—, —(CH₂)₁OC(O)(CH₂)ₕR*—, —(CH₂)₁CH═N(CH₂)ₕR*—, —(CH₂)₁N═CH(CH₂)ₕR*—, —(CH₂)₁NH(CH₂)ₕR*—, —(CH₂)₁S—(CH₂)ₕR*—, —(CH₂)₁O(CH₂)ₕR*— or —(CH₂)₁C(O)(CH₂)ₕR*—.

R* in each occurrence, independently is —CH₃ or —H.

In certain particular embodiments the reaction is carried out in a suitable solvents such as, for example, tetrahydrofuran, dichloromethane and toluene

In certain other particular embodiments the reaction is carried out in the presence of a suitable catalysts such as, for example, potassium carbonate, potassium hydroxide and sodium hydroxide.

In certain other particular embodiments the reaction is carried out under reflux conditions.

In certain other particular embodiments the reaction is carried out under a nitrogen atmosphere.

In certain other particular embodiments, the reaction is carried out at a temperature between about 50° C. and about 200° C. In certain other particular embodiments, the reaction is carried out at a temperature between about 80° C. and about 150° C. In certain other particular embodiments, the reaction is carried out at a temperature between about 100° C. and about 130° C.

In certain other particular embodiments, the reaction is carried for between 5 minutes and 60 hours, between 30 minutes and 36 hours, between 1 hours and 24 hours and between 2 hours and 12 hours.

In certain embodiments, macromolecular antioxidants of Structural Formulas I-VI are synthesized by reacting a compound represented R₁⁺⁺ represented by the following structural formula: 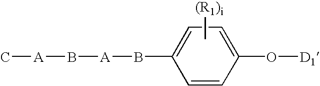
with a compound selected from:

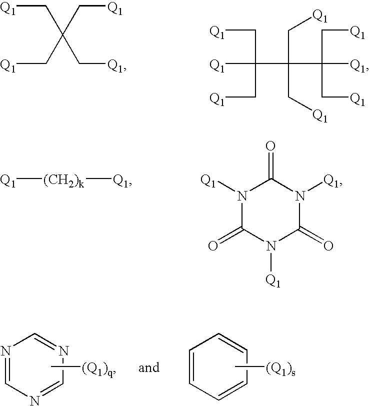

D₁′ is D₁ₐ′, D_{1b}′, D_{1c}′ or D_{1d}′;

Q₁ is Q₁ₐ or Q_{1b}; wherein when D₁′ is D₁ₐ′ or D_{1c}′, Q₁ is Q₁ₐ and when D₁′ is D_{1b}′ or D_{1d}′, Q₁ is Q_{1b}.

D₁ₐ′ is —(CH₂)₁C(O)—X and X is H or a leaving group. In a specific embodiment, X is H. In another specific embodiment, X is a halogen or —ORₑ, wherein Rₑ is an alkyl 10 group. In a more specific embodiment, X is —Cl or —Br. In another more specific embodiment, X is —ORₑ. Rₑ is preferably -Me.

D_{1b}′ is H, —(CH₂)₁NH₂, —(CH₂)₁SH, or —(CH₂)₁OH.

D₁ₑ′ is —(CH₂)₁NHC(O)(CH₂)ₕ—X′, —(CH₂)₁C(O)NH(CH₂)ₕ—X′, —(CH₂)₁C(O)O(CH₂)ₕ—X′, —(CH₂)₁OC(O)(CH₂)ₕ—X′, —(CH₂)₁CH═N(CH₂)ₕ—X′, —(CH₂)₁N═CH(CH₂)ₕ—X′, —(CH₂)₁NH(CH₂)ₕ—X′, —(CH₂)₁S—(CH₂)ₕ—X′, —(CH₂)₁O(CH₂)ₕ—X′ or —(CH₂)₁C(O)(CH₂)ₕ—X′, wherein h is not 0 and X′ is a leaving group. More specifically, X′ is a halogen.

D_{1d}′ is —(CH₂)₁NHC(O)(CH₂)ₕ—X″, —(CH₂)₁C(O)NH(CH₂)ₕ—X″, —(CH₂)₁C(O)O(CH₂)ₕ—X″, —(CH₂)₁OC(O)(CH₂)ₕ—X″, —(CH₂)₁CH═N(CH₂)ₕ—X″, —(CH₂)₁N═CH(CH₂)ₕ—X″, —(CH₂)₁NH(CH₂)ₕ—X″, —(CH₂)₁S—(CH₂)ₕ—, X″, —(CH₂)₁O(CH₂)ₕ—X″ or —(CH₂)₁C(O)(CH₂)ₕ—X″, wherein X″ is a nucleophile. More specifically, X″ is —NH₂ or —OH.

Q₁ₐ is a nucleophile. More specifically, Q_{1b} is —NH₂ or —OH.

Q_{1b} is a —W—X₁, wherein X₁ is a leaving group and W is, a bond or —C(O)—.

The remainder of the variables are as described above.

In a more specific embodiment, R₁⁺⁺is represented by the following structural formula A1′:

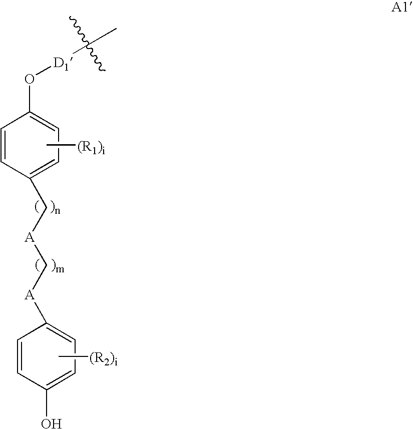

wherein D₁′ is as described above and the remainder of the variables are as defined as for Structural Formula A1.

In another more specific embodiment, R₁⁺⁺ is represented by the following structural formula A2′

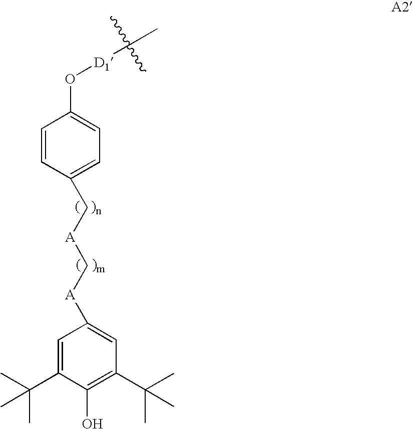

wherein D₁′ is as described above and the remainder of the variables are as defined as for Structural Formula A2.

In another more specific embodiment, R₁⁺⁺ is represented by the following structural formula A3′ 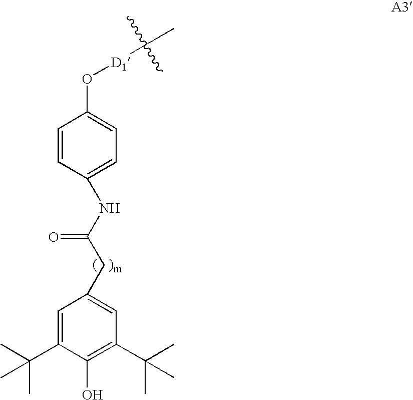
wherein D₁′ is as described above and the remainder of the variables are as defined as for Structural Formula A3.

In another more specific embodiment, R₁⁺⁺ is represented by the following structural formula A4′

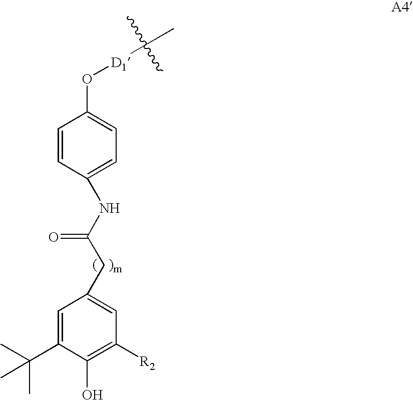

wherein D₁′ is as described above and the remainder of the variables are as defined as for Structural Formula A4.

In certain embodiments, when R₁⁺⁺ is represented by A1′, A2′, A3′ and A4′, D₁′ is D₁ₐ′ and Q₁ is Q₁ₐ. In a more specific embodiment, X is H and Q, is —NH₂. In another more specific embodiment, X is a halogen or —ORₑ, wherein Rₑ is an alkyl group and Q₁ is —NH₂ or —OH. Even more specifically, X is —Cl, —Br, or —OMe.

In certain embodiments, when R₁⁺⁺ is represented by A1′, A2′, A3′ and A4′, D₁′ is D_{1b}′ and Q₁ is Q_{1b}. In a more specific embodiment, W is a bond and X₁ is a halogen. In another more specific embodiment, W is —C(O)— and X₁ is a halogen or —ORₑ, wherein Rₑ is an alkyl group. Even more specifically, X is —Cl, —Br, or —OMe.

In certain embodiments, when R₁⁺⁺ is represented by A1′, A2′, A3′ and A4′, D₁′ is D_{1c}′ and Q₁ is Q₁ₐ. In a more specific embodiment, X′ is a halogen and Q₁ₐ is —NH₂ or —OH.

In certain embodiments, when R₁⁺⁺ is represented by A1′, A2′, A3′ and A4′, D₁′ is D_{1d}′ and Q₁ is Q_{1b}. In a more specific embodiment, X″ is —NH₂ or —OH. In a even more specific embodiment, W is a bond and X₁ is a halogen. In another even more specific embodiment, W is —C(O)— and X₁ is a halogen or —ORₑ, wherein Rₑ is an alkyl group. Even more specifically, X is —Cl, —Br, or —OMe.

In certain embodiments, macromolecular antioxidants of Structural Formulas I-VI are synthesized by reacting a compound represented R₂⁺⁺ represented by the following structural formula: 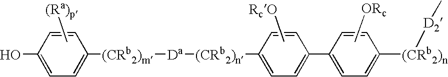
with a compound selected from: 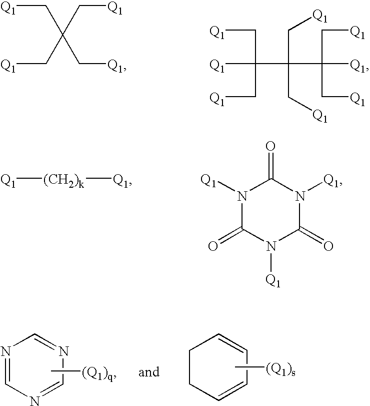
wherein D₂′ is D₂ₐ′ or D_{2b}′;

Q₁ is Q₁ₐ or Q_{1b}; wherein when D₂′ is D₂ₐ′, Q₁ is Q₁ₐ and when D₂′ is D_{2b′, Q}₁ is Q_{1b}.

D₂ₐ′ is —C(O)—X and X is H or a leaving group. In a specific embodiment, X is H. In another specific embodiment, X is a halogen or —ORₑ, wherein Rₑ is an alkyl group. In a more specific embodiment, X is —Cl or —Br. In another more specific embodiment, X is —ORₑ. Rₑ is preferably -Me.

D_{2b}′ is NHR_{d}, —SH, or —OH, wherein R_{d} is H or optionally substituted alkyl.

Q₁ₐ is a nucleophile. More specifically, Q_{1b} is —NH₂ or —OH.

Q_{1b} is a —W—X₁, wherein X₁ is a leaving group and W is a bond or —C(O)—.

In certain embodiments, R₂⁺⁺ is represented by the following structural formula: 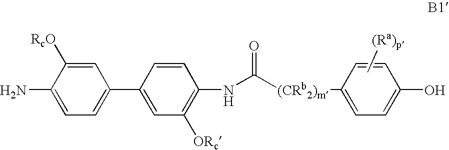
and Q₁ is a —W—X₁, wherein X₁ is a leaving group and W is a bond or —C(O)—. The remainder of the variable and their specific values are as described for Structural Formula B1. In a more specific embodiment, W is a bond and X₁ is a halogen. In another more specific embodiment, W is —C(O)—X₁ and X₁ is a halogen or —ORₑ, wherein Rₑ is an alkyl. Preferably, Rₑ is methyl.

In certain embodiments, R₂⁺⁺ is represented by the following structural formula: 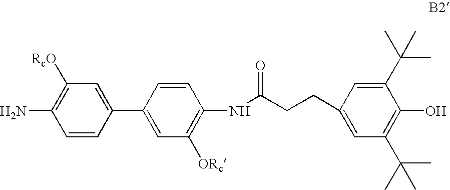
and Q₁ is a —W—X₁, wherein X₁ is a leaving group and W is a bond or —C(O)—. The remainder of the variable and their specific values are as described for Structural Formula B2. In a more specific embodiment, W is a bond and X₁ is a halogen. In another more specific embodiment, W is —C(O)—X₁ and X₁ is a halogen or —ORₑ, wherein Rₑ is an alkyl. Preferably, Rₑ is methyl.

Examples of reactions for synthesizing macromolecular antioxidants of Structural Formulas I-VI, where R is represented by Structural Formula A, are illustrated in the following schemes:

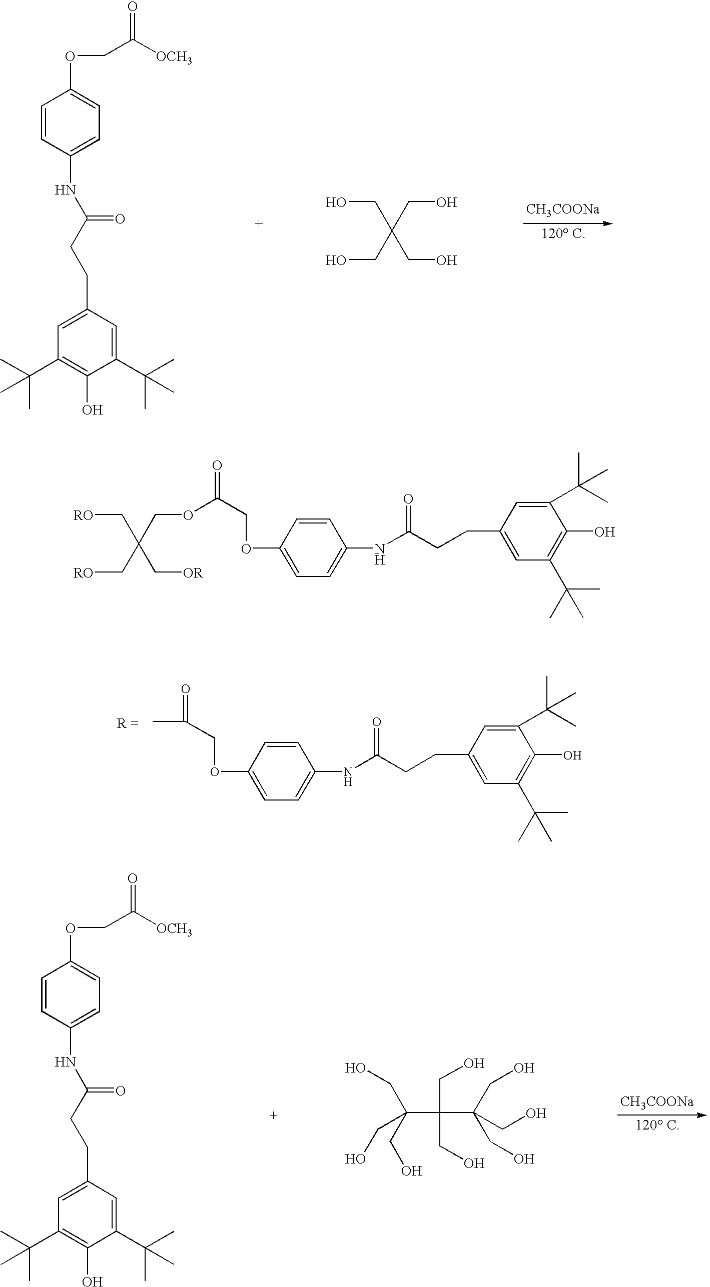

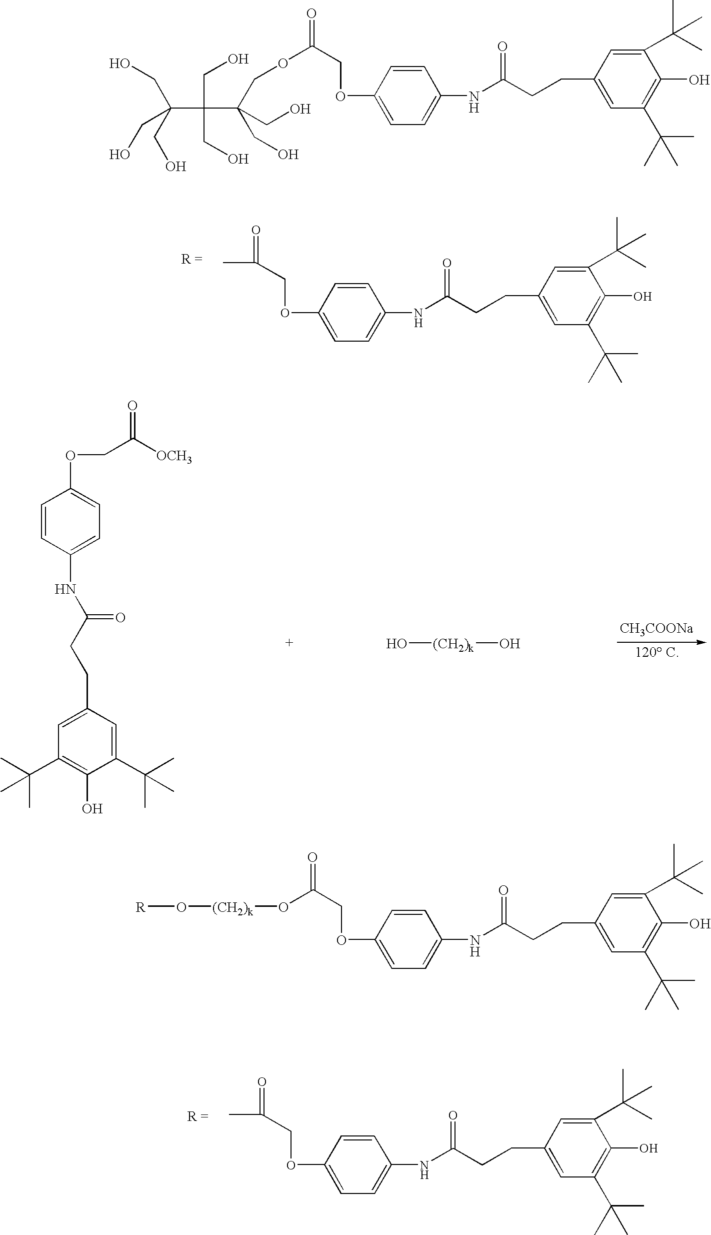

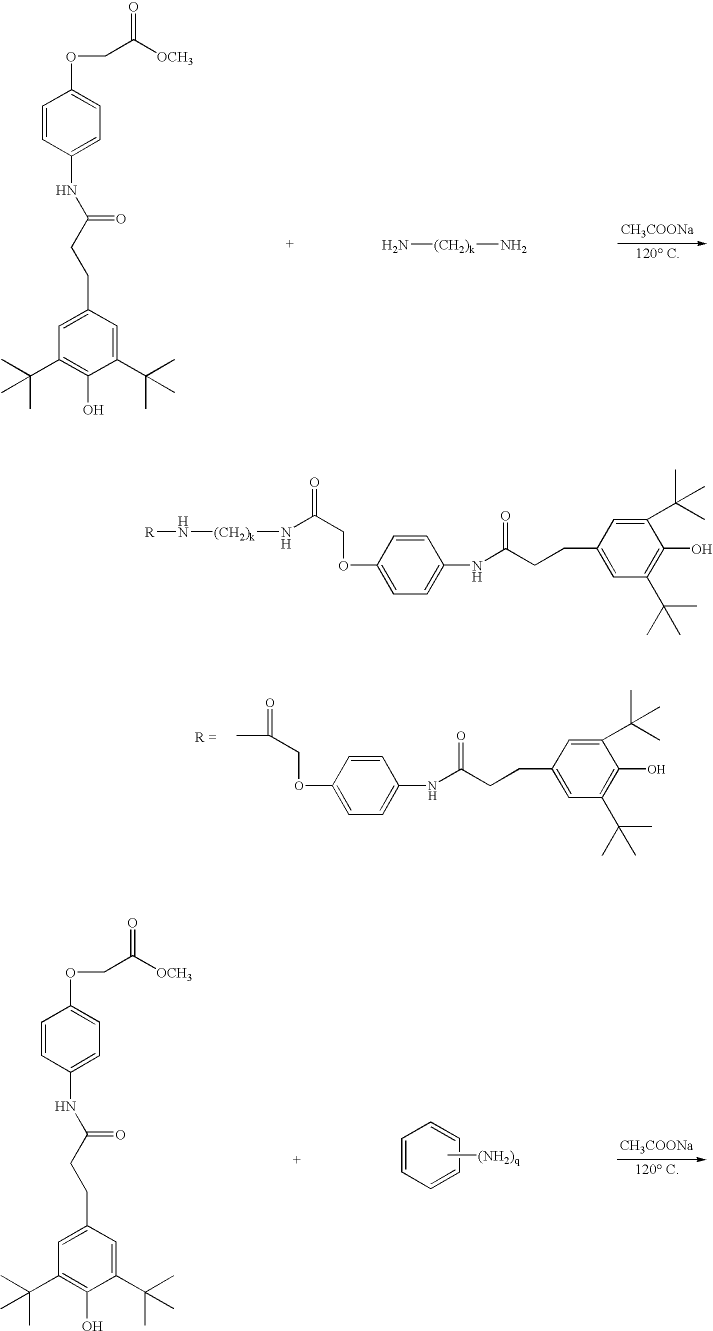

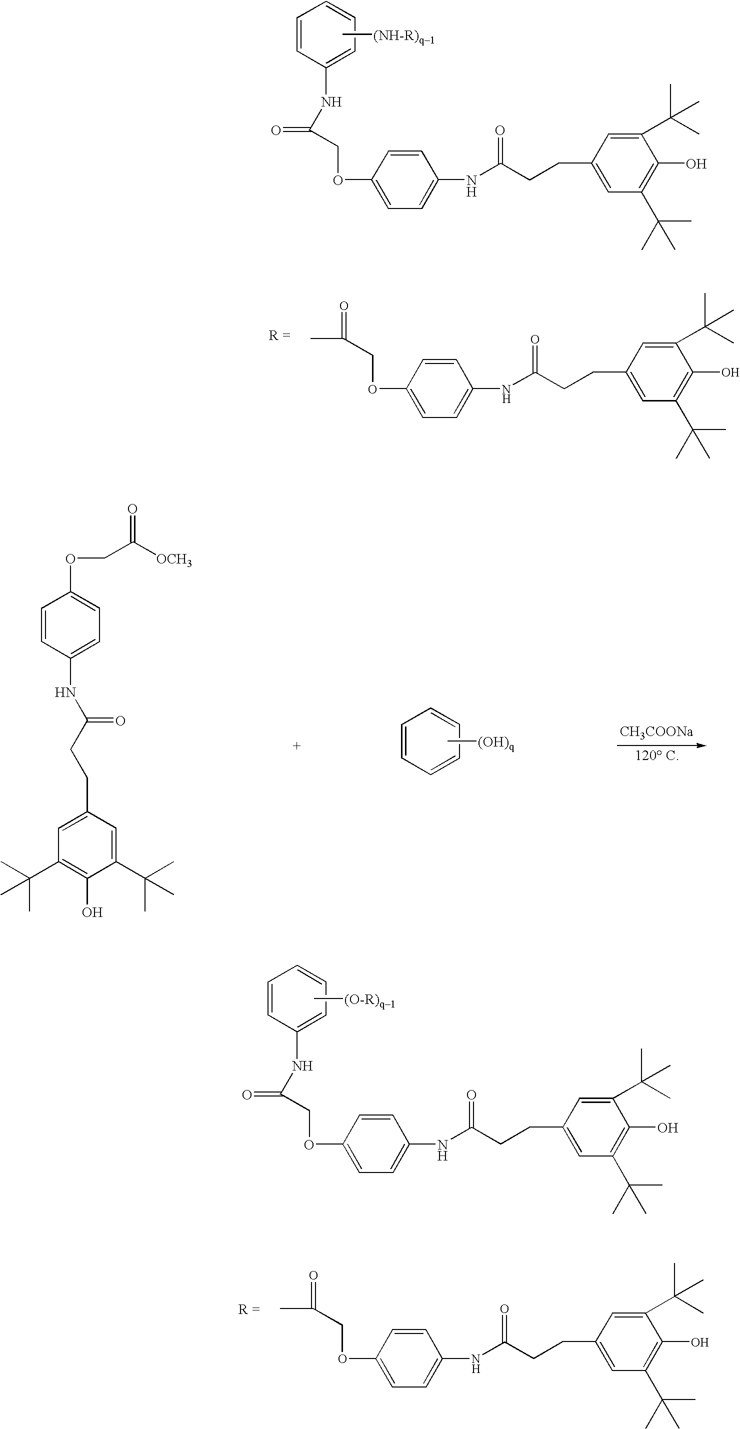

In certain other particular embodiments the reaction is carried out without solvent in bulk reaction conditions using a suitable catalyst such as, for example, sodium acetate or lithium carbonate.

In certain other particular embodiments the reaction is carried out under melt conditions or in heterogeneous melt.

In certain other particular embodiments the reaction is carried out under a nitrogen atmosphere or under vacuum.

In certain other particular embodiments, the reaction is carried out at a temperature between about 50° C. and about 200° C. In certain other particular embodiments, the reaction is carried out at a temperature between about 80° C. and about 150° C. In certain other particular embodiments, the reaction is carried out at a temperature between about 100° C. and about 130° C.

In certain other particular embodiments, the reaction is carried for between 30 minutes and 96 hours, between 1 hour and 72 hours, between 2 hours and 60 hours between 4 hours and 48 hours and between 12 hours and 24 hours.

In certain embodiments, D′ as defined above acts as a linker group which can act as a remote handle in coupling of an R group to form the macromolecular antioxidant of the present invention. The addition of a linker D′ to a phenol can be carried out in a similar fashion as shown below:

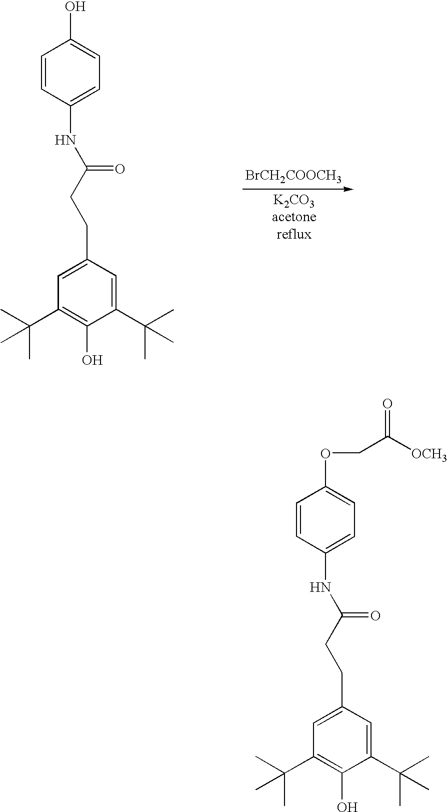

In certain particular embodiments the reaction is carried out in a suitable solvent such as, for example, acetone, acetonitrile and tetrahydrofuran.

In certain other particular embodiments the reaction is carried out in the presence of a suitable catalysts such as, for example, potassium carbonate and sodium carbonate.

In certain other particular embodiments the reaction is carried out under reflux conditions

In certain other particular embodiments the reaction is carried out under a nitrogen atmosphere

In certain other particular embodiments, the reaction is carried out at a temperature between about 5° C. and about 200° C. In certain other particular embodiments, the reaction is carried out at a temperature between about 10° C. and about 150° C. In certain other particular embodiments, the reaction is carried out at a temperature between about 50° C. and about 100° C. In certain other particular embodiments, the reaction is carried out at a temperature between about 60° C. and about 80° C.

In certain other particular embodiments, the reaction is carried out for between 30 minutes and 72 hours, between 1 hour and 48 hours, between 2 hours and 24 hours between 4 hours and 18 hours and between 10 hours and 14 hours.

Examples of reactions for synthesizing macromolecular antioxidants of Structural Formula I-VI, wherein R is represented by Structural Formula B are shown in the following schemes: 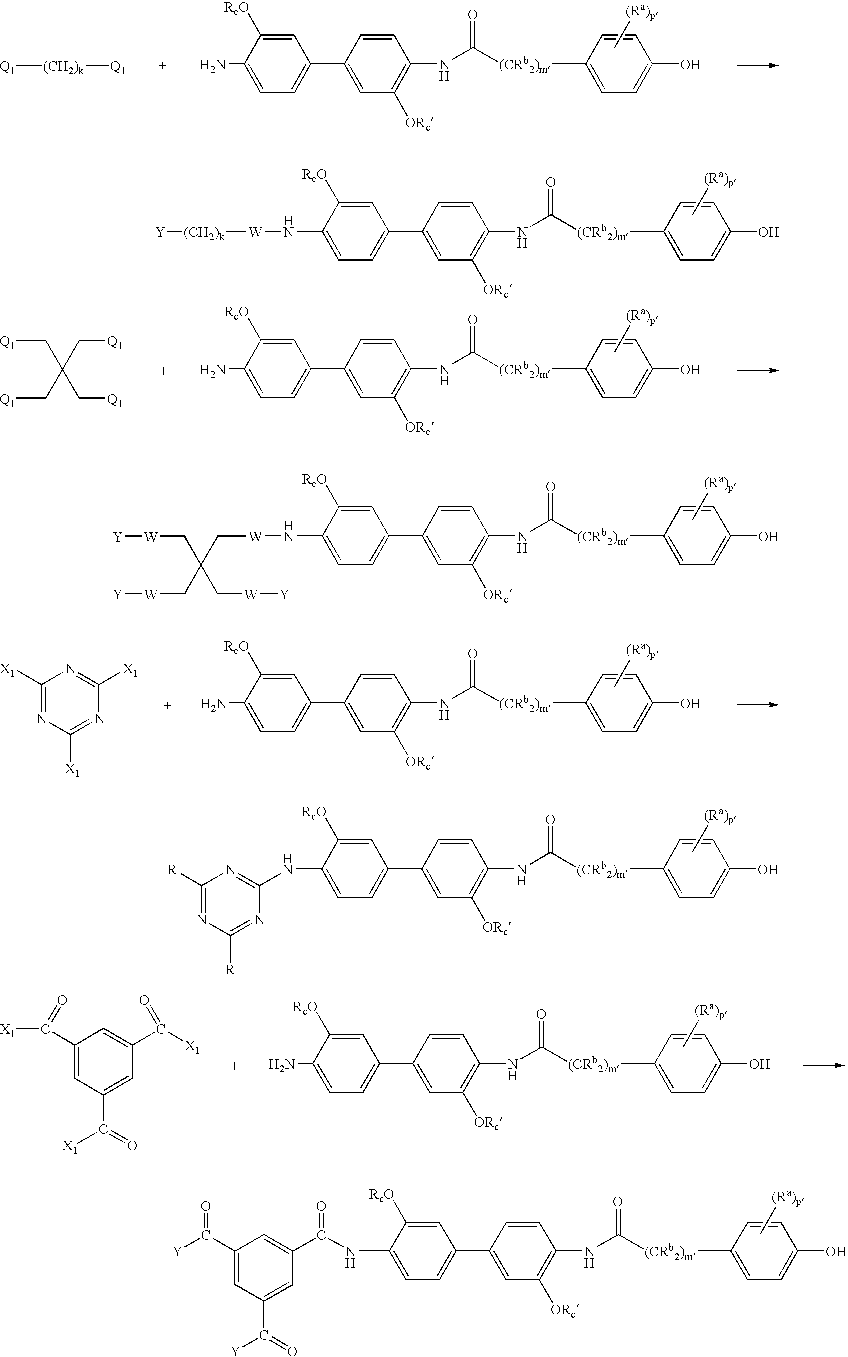
wherein:

Y is represented by the following structural formula:

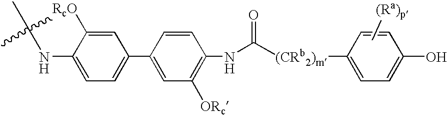

Q₁ is —W—X₁;

W is a bond or —C(O); and

X₁ is a leaving group. More specifically, X₁ is a halogen.

Macromolecular antioxidants of Structural Formula I-VI of the present invention can be prepared in a similar fashion according to the reactions described above.

In certain particular embodiments, the present invention pertains to polymeric macromolecular antioxidants comprising at least one repeating unit represented by VIIa, VIIb, VIIIa, VIIIb or a combination thereof:

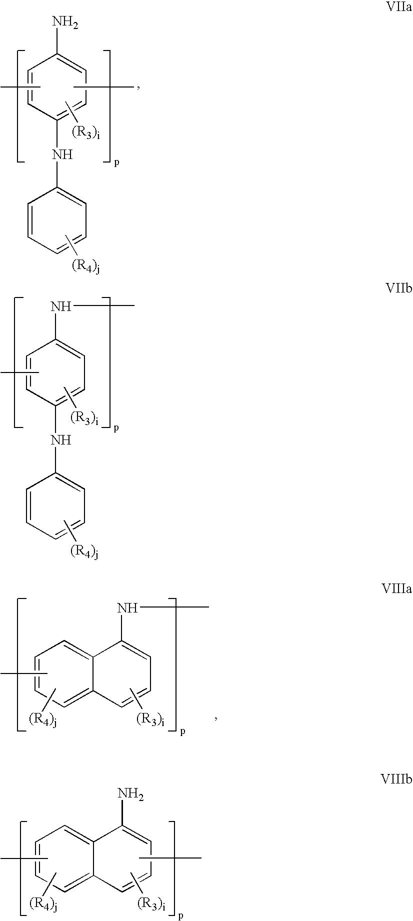

R₃ and R₄ in each occurrence, independently is C1-C16 alkyl, —O—(C1-C16 alkyl), —NH(aryl), —NH₂, —OH, or —SH.

p in each occurrence, independently is an integer equal to or greater than 2.

In another particular embodiment, the present invention pertains to a method for the synthesis of polymeric macromolecular antioxidants containing aromatic amine type antioxidant units where antioxidant units are, for example, but not limited to C-substituted anilines/dianilines, C-substituted napthylamines, N-substituted anilines/dianilines, N-substituted napthylamines and their combination in various ratios. In certain embodiments, the macromolecular antioxidants contain both C—N—C and C—C couplings in the backbone.

In certain embodiments, the process involves the polymerization of aromatic amine type monomeric system such as C-substituted anilines/dianilines, C-substituted napthylamines, N-substituted anilines/dianilines, N-substituted napthylamines, their combination in various ratios and other active aromatic amines leading to the formation of polymeric macromolecular antioxidants.

In one example, the polymeric macromolecular antioxidant based on N-substituted anilines/dianilines type monomer may contain Structures VIIa, VIIb or both.

In another example, the polymeric macromolecule based on napthylamine type monomer may contain Structures VIIIa, VIIIb or both.

In certain embodiments, the present invention pertains to methods of synthesizing polymer represented by structural formulas VIIa, VIIb, VIIIa and VIIIb.

In certain other embodiments these polymers are synthesized by polymerizing a monomer represented by a structural formula selected from: 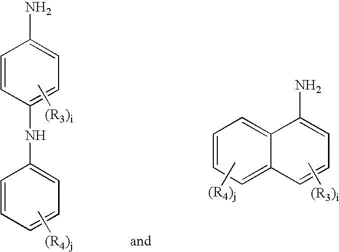
or combinations thereof using an oxidative polymerization catalyst.

In certain embodiments, the oxidative polymerization catalyst is a biocatalyst or a biomimetic catalyst selected from Iron(II)-salen complexes, horseradish peroxidase, soybean peroxidase, hematin, laccase, tyroniase, ferric chloride and ammonium persulphate and a tyroniase-model complex.

In certain other embodiments, the oxidative polymerization catalyst is an inorganic or organometallic catalyst.

In yet another particular embodiment, the present invention pertains to a method for the synthesis of polymeric macromolecules, where catalysts used for polymerization are for example, but not limited to enzyme or enzyme mimetic catalysts.

Examples of enzyme or enzyme mimetic used for polymerization include Iron(II)-salen complexes, horseradish peroxidase (HRP), soybean peroxidase (SBP), hematin, laccase, tyroniase, tyroniase-model complexes and other peroxidases.

In yet another particular embodiment, the present invention relates to a simple process for the synthesis of polymeric macromolecular antioxidants based on aromatic amine type antioxidant units using typical biocatalysts such as peroxidases e.g. horse radish peroxidase (HRP), biomimetic type catalysts (e.g. hematin) or other inorganic catalysts such as Fe-Salen.

In certain embodiments the polymeric antioxidants of the present invention are synthesized as follows:

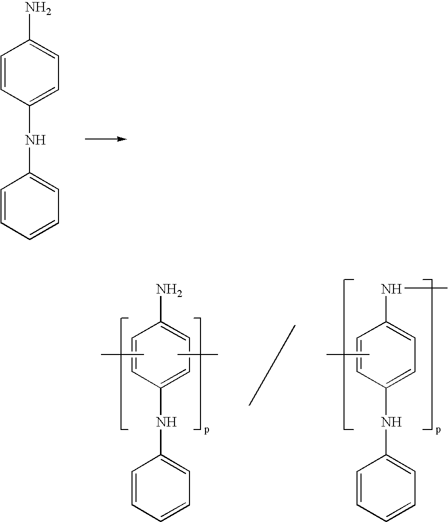

In certain other particular embodiments the reaction is carried out in the presence of a suitable solvent such as, for example, tetrahydrafuma (THF), dioxane, acetonitrile, DMF and methanol.

In certain embodiments, the reaction is carried out in the presence of an oxidative polymerization catalyst selected from Iron(II)-salen complexes, horseradish peroxidase, soybean peroxidase, hematin, laccase, tyroniase, ferric chloride and ammonium persulphate and a tyroniase-model complex.

In certain other particular embodiments, the reaction is carried out at a temperature between about 2° C. and about 120° C. In certain other particular embodiments, the reaction is carried out at a temperature between about 5° C. and about 100° C. In certain other particular embodiments, the reaction is carried out at a temperature between about 10° C. and about 60° C. In certain other particular embodiments, the reaction is carried out at a temperature between about 20° C. and about 40° C.

In certain other particular embodiments, the reaction is carried for between 30 minutes and 96 hours, between 1 hour and 72 hours, between 2 hours and 60 hours between 4 hours and 48 hours and between 12 hours and 24 hours.

In certain other embodiments the polymeric antioxidants of the present invention are synthesized as follows:

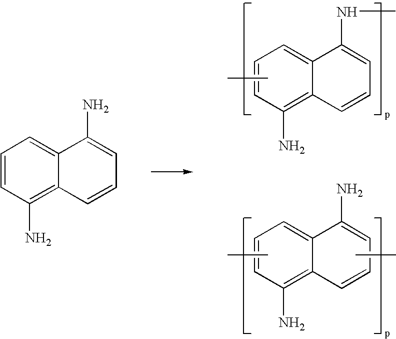

In certain other particular embodiments the reaction is carried out in the presence of a suitable solvent such as, for example, tetrahydrafuma (THF), dioxane, methanol, diimethylformamide (DMF) and acetonitrile

In certain embodiments, the reaction is carried out in the presence of an oxidative polymerization catalyst selected from Iron(II)-salen complexes, horseradish peroxidase, soybean peroxidase, hematin, laccase, tyroniase, ferric chloride, ammonium persulfate and a tyroniase-model complex.

In certain other particular embodiments, the reaction is carried out at a temperature between about 2° C. and about 120° C. In certain other particular embodiments, the reaction is carried out at a temperature between about 5° C. and about 100° C. In certain other particular embodiments, the reaction is carried out at a temperature between about 10° C. and about 60° C. In certain other particular embodiments, the reaction is carried out at a temperature between about 20° C. and about 40° C.

In certain other particular embodiments, the reaction is carried for between 30 minutes and 96 hours, between 1 hour and 72 hours, between 2 hours and 60 hours between 4 hours and 48 hours and between 12 hours and 24 hours.

The term “alkyl” as used herein means a saturated straight-chain, branched or cyclic hydrocarbon. When straight-chained or branched, an alkyl group is typically C1-C8, more typically C1-C6; when cyclic, an alkyl group is typically C3-C12, more typically C3-C7 alkyl ester. Examples of alkyl groups include methyl, ethyl, n-propyl, iso-propyl, n-butyl, sec-butyl and tert-butyl and 1,1-dimethylhexyl.

The term “alkoxy” as used herein is represented by —OR**, wherein R** is an alkyl group as defined above.

The term “acyl” as used herein is represented by —C(O)R**, wherein R** is an alkyl group as defined above.

The term “alkyl ester” as used herein means a group represented by —C(O)OR**, where R** is an alkyl group as defined above.

The term “aromatic group” used alone or as part of a larger moiety as in “aralkyl”, includes carbocyclic aromatic rings and heteroaryl rings. The term “aromatic group” may be used interchangeably with the terms “aryl”, “aryl ring” “aromatic ring”, “aryl group” and “aromatic group”.

Carbocyclic aromatic ring groups have only carbon ring atoms (typically six to fourteen) and include monocyclic aromatic rings such as phenyl and fused polycyclic aromatic ring systems in which a carbocyclic aromatic ring is fused to one or more aromatic rings (carbocyclic aromatic or heteroaromatic). Examples include 1-naphthyl, 2-naphthyl, 1-anthracyl and 2-anthracyl. Also included within the scope of the term “carbocyclic aromatic ring”, as it is used herein, is a group in which an aromatic ring is fused to one or more non-aromatic rings (carbocyclic or heterocyclic), such as in an indanyl, phthalimidyl, naphthimidyl, phenanthridinyl, or tetrahydronaphthyl, where the radical or point of attachment is on the aromatic ring.

The term “heteroaryl”, “heteroaromatic”, “heteroaryl ring”, “heteroaryl group” and “heteroaromatic group”, used alone or as part of a larger moiety as in “heteroaralkyl” refers to heteroaromatic ring groups having five to fourteen members, including monocyclic heteroaromatic rings and polycyclic aromatic rings in which a monocyclic aromatic ring is fused to one or more other aromatic ring (carbocyclic aromatic or heteroaromatic). Heteroaryl groups have one or more ring heteroatoms. Examples of heteroaryl groups include 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 3-pyrazolyl, 4-pyrazolyl, 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 3-pyridazinyl, 2-thiazolyl, 4-thiazolyl, 5-thiazolyl, 2-triazolyl, 5-triazolyl, tetrazolyl, 2-thienyl, 3-thienyl, carbazolyl, 2-benzothienyl, 3-benzothienyl, 2-benzofuranyl, 3-benzofuranyl, 2-indolyl, 3-indolyl, 2-quinolinyl, 3-quinolinyl, 2-benzothiazole, 2-benzooxazole, 2-benzimidazole, 2-quinolinyl, 3-quinolinyl, 1-isoquinolinyl, 3-quinolinyl, 1-isoindolyl and 3-isoindolyl. Also included within the scope of the term “heteroaryl”, as it is used herein, is a group in which an aromatic ring is fused to one or more non-aromatic rings (carbocyclic or heterocyclic), where the radical or point of attachment is on the aromatic ring.

The term “heteroatom” means nitrogen, oxygen, or sulfur and includes any oxidized form of nitrogen and sulfur, and the quatemized form of any basic nitrogen. Also the term “nitrogen” includes a substitutable nitrogen of a heteroaryl or non-aromatic heterocyclic group. As an example, in a saturated or partially unsaturated ring having Q-3 heteroatoms selected from oxygen, sulfur or nitrogen, the nitrogen may be N (as in 3,4-dihydro-2H-pyrrolyl), NH (as in pyrrolidinyl) or NR″ (as in N-substituted pyrrolidinyl), wherein R″ is a suitable substituent for the nitrogen atom in the ring of a non-aromatic nitrogen-containing heterocyclic group, as defined below.

An “aralkyl group”, as used herein is an alkyl groups substituted with an aryl group as defined above.

An optionally substituted aryl group as defined herein may contain one or more substitutable ring atoms, such as carbon or nitrogen ring atoms. Examples of suitable substituents on a substitutable ring carbon atom of an aryl group include —OH, C1-C3 alkyl, C1-C3 haloalkyl, —NO₂, C1-C3 alkoxy, C1-C3 haloalkoxy, —CN, —NH₂, C1-C3 alkylamino, C1-C3 dialkylamino, —C(O)NH₂, —C(O)NH(C1-C3 alkyl), —C(O)(C1-C3 alkyl), —NHC(O)H, —NHC(O)(C1-C3 alkyl), —C(O)N(C1-C3 alkyl)₂, —NHC(O)OC1-C3 alkyl), —C(O)OH, —C(O)O—(C1-C3 alkyl), —NHC(O)NH₂, —NHC(O)NH(C1-C3 alkyl), —NHC(O)N(C1-C3 alkyl)₂, —SO₂NH₂—SO₂NH(C1-C3alkyl), —SO₂N(C1-C3alkyl)₂, NHSO₂H or NHSO₂(C 1-C3 alkyl). Preferred substituents on aryl groups are as defined throughout the specification. In certain embodiments optionally substituted aryl groups are unsubstituted

Examples of suitable substituents on a substitutable ring nitrogen atom of an aryl group include C1-C3 alkyl, NH₂, C1-C3 alkylamino, C1-C3 dialkylamino, —C(O)NH₂, —C(O)NH(C1-C3 alkyl), —C(O)(C1-C3 alkyl), —CO₂R**, —C(O)C(O)R**, —C(O)CH₃, —C(O)OH, —C(O)O—(C1-C3 alkyl), —SO₂NH₂—SO₂NH(C1-C3alkyl), —SO₂N(C1-C3alkyl)₂, NHSO₂H, NHSO₂(C1-C3 alkyl), —C(═S)NH₂, —C(═S)NH(C1-C3 alkyl), —C(═S)N(C1-C3 alkyl)₂, —C(═NH)—N(H)₂, —C(═NH)—NH(C1-C3 alkyl) and —C(═NH)—N(C1-C3 alkyl)₂,

An optionally substituted alkyl group as defined herein may contain one or more substituents. Examples of suitable substituents for an alkyl group include those listed above for a substitutable carbon of an aryl and the following: ═O, ═S, ═NNHR**, ═NN(R**)₂, ═NNHC(O)R**, ═NNHCO₂ (alkyl), ═NNHSO₂(alkyl), ═NR**, spiro cycloalkyl group or fused cycloalkyl group. R** in each occurrence, independently is —H or C1-C6 alkyl. Preferred substituents on alkyl groups are as defined throughout the specification. In certain embodiments optionally substituted alkyl groups are unsubstituted.

A “spiro cycloalkyl” group is a cycloalkyl group which shares one ring carbon atom with a carbon atom in an alkylene group or alkyl group, wherein the carbon atom being shared in the alkyl group is not a terminal carbon atom.

A “leaving group” is a group which can readily be displaced by a nucleophile. Examples of a good leaving group include but not limited halogen, alkoxy group and a tosylate group.

A “nucleophile” is a reagent that brings an electron pair. Typical nucleophile include but not limited amines and alcohols.

Without wishing to be bound by any theory or limited to any mechanism it is believed that macromolecular antioxidants and polymeric macromolecular antioxidants of the present invention exploit the differences in activities (ks, equilibrium constant) of, for example, homo- or hetero-type antioxidant moieties. Antioxidant moieties include, for example, hindered phenolic groups, unhindered phenolic groups, aminic groups and thioester groups, etc. of which there can be one or more present in each macromolecular antioxidant molecule. As used herein a homo-type antioxidant macromolecule comprises antioxidant moieties which are all same, for example, hindered phenolic, —OH groups. As used herein a hetero-type antioxidant macromolecule comprises at least one different type of moiety, for example, hindred phenolic and aminic groups in the one macromolecule.

This difference in activities can be the result of, for example, the substitutions on neighboring carbons or the local chemical or physical environment (for example, due to electrochemical or stereochemical factors) which can be due in part to the macromolecular nature of molecules.

In one embodiment of the present invention, a series of macromolecular antioxidant moieties of the present invention with different chemical structures can be represented by W1H, W2H, W3H, . . . to WnH. In one embodiment of the present invention, two types of antioxidant moieties of the present invention can be represented by: W1H and W2H. In certain embodiments W1H and W2H can have rate constants of k1 and k2 respectively. The reactions involving these moieties and peroxyl radicals can be represented as: 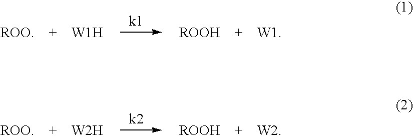
where ROO. is a peroxyl radical resulting from, for example, initiation steps involving oxidation activity, for example:
RH→R.+H. (3)
R.+O2→ROO. (4)

In one particular embodiment of the present invention k1>>k2 in equations (1) and (2). As a result, the reactions would take place in such a way that there is a decrease in concentration of W1. free radicals due their participation in the regeneration of active moiety W2H in the molecule according equation (5):
W1.+W2H→W1H+W2. (5) (transfer equilibrium)

This transfer mechanism may take place either in intra- or inter-molecular macromolecules. The transfer mechanism (5) could take place between moieties residing on the same macromolecule (intra-type) or residing on different macromolecules (inter-type).

In certain embodiments of the present invention, the antioxidant properties described immediately above (equation 5) of the macromolecular antioxidants and polymeric macromolecular antioxidants of the present invention result in advantages including, but not limited to:
a) Consumption of free radicals W1. according to equation (5) can result in a decrease of reactions of W1. with hydroperoxides and hydrocarbons (RH).
b) The regeneration of W1H provides extended protection of materials. This is a generous benefit to sacrificial type of antioxidants that are used today. Regeneration of W1H assists in combating the oxidation process The increase in the concentration of antioxidant moieties W1H (according to equation 5) extends the shelf life of materials.

In certain embodiments of the present invention, the following items are of significant interest f6r enhanced antioxidant activity in the design of the macromolecular antioxidants and polymeric macromolecular antioxidants of the present invention:
a) The activity of proposed macromolecular antioxidant is dependent on the regeneration of W1H in equation (5) either through inter- or intra-molecular activities involving homo- or hetero-type antioxidant moieties.
b) Depending on the rates constants of W1H and W2H it is possible to achieve performance enhancements by many multiples and not just incremental improvements.

In certain embodiments of the present invention, more than two types of antioxidant moieties with different rate constants are used in the methods of the present invention.

In certain embodiments, the present invention pertains to the use of the disclosed compounds to inhibit oxidation in an oxidizable material. This process involves contact the oxidizable material with a compound or polymer of the present invention.

For purposes of the present invention, a method of “inhibiting oxidation” is a method that inhibits the propagation of a free radical-mediated process. Free radicals can be generated by heat, light, ionizing radiation, metal ions and some proteins and enzymes. Inhibiting oxidation also includes inhibiting reactions caused by the presence of oxygen, ozone or another compound capable of generating these gases or reactive equivalents of these gases.

As used herein the term “oxidizable material” is any material which is subject to oxidation by free-radicals or oxidative reaction caused by the presence of oxygen, ozone or another compound capable of generating these gases or reactive equivalents thereof.

Antioxidant compounds and polymers of the present invention can be used to prevent oxidation in a wide variety of compositions where free radical mediated oxidation leads to deterioration of the quality of the composition, including edible products such as oils, foods (e.g., meat products, dairy products, cereals, etc.), and other products containing fats or other compounds subject to oxidation. Antioxidant compounds and polymers-can also be present in plastics and other polymers, elastomers (e.g., natural or synthetic rubber), petroleum products (e.g., fossil fuels such as gasoline, kerosene, diesel oil, heating oil, propane, jet fuel), lubricants, paints, pigments or other colored items, soaps and cosmetics (e.g., creams, lotions, hair products). The antioxidant compounds and polymers can be used to coat a metal as a rust and corrosion inhibitor. Antioxidant compounds and polymers additionally can protect antioxidant vitamins (Vitamin A, Vitamin C, Vitamin E) and pharmaceutical products from degradation. In food products, the antioxidant compounds can prevent rancidity. In plastics, the antioxidant compounds and polymers can prevent the plastic from becoming brittle and cracking.

Antioxidant compounds and polymers of the present invention can be added to oils to prolong their shelf life and properties. These oils can be formulated as vegetable shortening or margarine. Oils generally come from plant sources and include cottonseed oil, linseed oil, olive oil, palm oil, corn oil, peanut oil, soybean oil, castor oil, coconut oil, safflower oil, sunflower oil, canola (rapeseed) oil and sesame oil. These oils contain one or more unsaturated fatty acids such as caproleic acid, palmitoleic acid, oleic acid, vaccenic acid, elaidic acid, brassidic acid, erucic acid, nervonic acid, linoleic acid, eleosteric acid, alpha-linolenic acid, gamma-linolenic acid, and arachidonic acid, or partially hydrogenated or trans-hydrogenated variants thereof. Antioxidant compounds and polymers of the present invention are also advantageously added to food or other consumable products containing one or more of these fatty acids.

The shelf life of many materials and substances contained within the materials, such as packaging materials, are enhanced by the presence of an antioxidant compound or polymer of the present invention. The addition of an antioxidant compound or polymer to a packaging material is believed to provide additional protection to the product contained inside the package. In addition, the properties of many packaging materials themselves, particularly polymers, are enhanced by the presence of an antioxidant regardless of the application (i.e., not limited to use in packaging). Common examples of packaging materials include paper, cardboard and various plastics and polymers. A packaging material can be coated with an antioxidant compound or polymer (e.g., by spraying the antioxidant polymer or by applying as a thin film coating), blended with or mixed with an antioxidant compound or polymer (particularly for polymers), or otherwise have an antioxidant polymer present within it. In one example, a thermoplastic such as polyethylene, polypropylene or polystyrene can be melted in the presence of an antioxidant polymer in order to minimize its degradation during the polymer processing. An antioxidant polymer can also be co-extruded with a polymeric material.

The entire teachings of each of the following applications are incorporated herein by reference:
Docket No.: 3805.1000-000; Provisional Patent Application No. 60/632,893, filed Dec. 3, 2004, Title: Process For The Synthesis Of Polyalkylphenol Antioxidants, by Suizhou Yang, et al;
Docket No.: 3805.1000-003; Patent Application Ser. No. 11/292,813, filed Dec. 2, 2005, Title: Process For The Synthesis Of Polyalkylphenol Antioxidants, by Shuzhou Yang, et al;
Docket No.: 3805.1001-000; Provisional Patent Application No. 60/633,197, filed Dec. 3, 2004, Title: Synthesis Of Sterically Hindered Phenol Based Macromolecular Antioxidants, by Ashish Dhawan, et al.;
Docket No.: 3805.1001-003; Patent Application Ser. No. 11/293,050, filed Dec. 2, 2005, Title: Synthesis Of Sterically Hindered Phenol Based Macromolecular Antioxidants, by Ashish Dhawan, et al.;
Docket No.: 3805.1002-000; Provisional Patent Application No. 60/633,252, filed Dec. 3, 2004, Title: One Pot Process For Making Polymeric Antioxidants, by Vijayendra Kumar, et al.;
Docket No.: 3805.1002-003; Patent Application Ser. No. 11/293,049, filed Dec. 2, 2005, Title: One Pot Process For Making Polymeric Antioxidants, by Vijayendra Kumar, et al.;
Docket No.: 3805.1003-000; Provisional Patent Application No. 60/633,196, filed Dec. 3, 2004, Title: Synthesis Of Aniline And Phenol-Based Macromonomers And Corresponding Polymers, by Rajesh Kumar, et al.;
Docket No.: 3805.1003-003; Patent Application Ser. No. 11/293,844, filed Dec. 2, 2005, Title: Synthesis Of Aniline And Phenol-Based Macromonomers And Corresponding Polymers, by Rajesh Kumar, et al.;
Docket No.: 3805.1004-000; Provisional Patent Application No. 60/590,575, filed Jul. 23, 2006, Title: Anti-Oxidant Macromonomers And Polymers And Methods Of Making And Using The Same, by Ashok L. Cholli;
Docket No.: 3805.1004-001; Provisional Patent Application No. 60/590,646, filed Jul. 23, 2006, Title: Anti-Oxidant Macromonomers And Polymers And Methods Of Making And Using The Same, by Ashok L. Cholli;
Docket No.: 3805.1004-002; Patent Application Ser. No. 11/184,724, filed Jul. 19, 2005, Title: Anti-Oxidant Macromonomers And Polymers And Methods Of Making And Using The Same, by Ashok L. Cholli;
Docket No.: 3805.1004-005; Patent Application Ser. No. 11/184,716, filed Jul. 19, 2005, Title: Anti-Oxidant Macromonomers And Polymers And Methods Of Making And Using The Same, by Ashok L. Cholli;
Docket No.: 3805.1005-000; Provisional Patent Application No. 60/655,169, filed Feb. 22, 2005, Title: Nitrogen And Hindered Phenol Containing Dual Functional Macromolecules: Synthesis And Their Antioxidant Performances In Organic Materials, by Rajesh Kumar, et al.
Docket No.: 3805.1005-003; Patent Application Ser. No. 11/360,020, filed Feb. 22, 2006, Title: Nitrogen And Hindered Phenol Containing Dual Functional Macromolecules: Synthesis, Performances And Applications, by Rajesh Kumar, et al.
Docket No.: 3805.1006-000; Provisional Patent Application No. 60/6625,638, filed Mar. 25, 2005, Title: Alkylated Macromolecular Antioxidants And Methods Of Making, And Using The Same, by Rajesh Kumar, et al.
Docket No.: 3805.1006-001; Patent Application Ser. No. 11/389,564, filed Mar. 24, 2006, Title: Alkylated Macromolecular Antioxidants And Methods Of Making, And Using The Same, by Rajesh Kumar, et al.
Docket No.: 3805.1008-000; Provisional Patent Application No. 60/731,021, filed Oct. 27, 2005, Title: Macromolecular Antioxidants Based On Sterically Hindered Phenols And Phosphites, by Ashok L. Cholli, et al.
Docket No.: 3805.1008-001; Patent Application, filed Oct. 27, 2006, Title: Macromolecular Antioxidants Based On Sterically Hindered Phenols And Phosphites, by Ashok L. Cholli, et al.
Docket No.: 3805.1009-000; Provisional Patent Application No. 60/742,150, filed Dec. 2, 2005, Title: Lubricant Oil Composition, by Ashok L. Cholli, et al.
Docket No.: 3805.1010-000; Provisional Patent Application No. 60/731,325, filed Oct. 27, 2005, Title: Stabilized Polyolefin Composition, by Vijayendra Kumar, et al.
Docket No.: 3805.1010-001; Patent Application, filed Oct. 27, 2006, Title: Stabilized Polyolefin Composition, by Vijayendra Kumar, et al.
Docket No.: 3805.1011-000; Provisional Patent Application No. 60/818,876, filed Jul. 6, 2006, Title: Novel Macromolecular Antioxidants Comprising Differing Antioxidant Moieties Structures Methods of Making and Using the Same, by Ashok L. Cholli, et al.
Docket No.: 0813.2006-003; Patent Application Ser. No. 11/040,193, filed Jan. 21 2005, Title: Post-Coupling Synthetic Approach For Polymeric Antioxidants, by Ashok L. Choll, et al.;
Docket No.: 0813.2006-002; Patent Application No.: PCT/US2005/001948, filed Jan. 21, 2005, Title: Post-Coupling Synthetic Approach For Polymeric Antioxidants, by Ashok L. Cholli et al.;
Docket No.: 0813.2002-008; Patent Application No.: PCT/US2005/001946, filed Jan. 21 2005, Title: Polymeric Antioxidants, by Ashok L. Choll, et al.;
Docket No.: 0813.2002-00 3, Patent Application No.: PCT/US03/10782, filed Apr. 4, 2003, Title: Polymeric Antioxidants, by Ashok L. Choll, et al.;
Docket No.: 0813.2002-004; Patent Application Ser. No. 10/761,933, filed Jan. 21, 2004, Title: Polymeric Antioxidants, by Ashish Dhawan, et al.;
Docket No.: 0813.2002-001; Patent Application Ser. No. 10/408,679, filed Apr. 4, 2003, Title: Polymeric Antioxidants, by Ashok L. Choll, et al.;
   Exemplification

### EXAMPLE 1

### Synthesis of 1,6-bis [N-(4-hydroxyphenyl)-3-(2,6-di-tert-butyl, 4-hydroxyphenyl)propionamide]hexyl ether

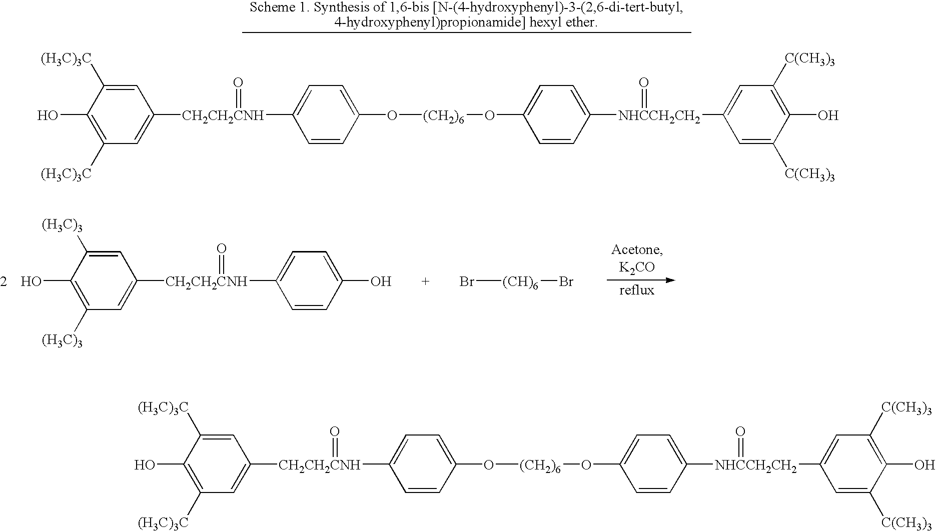

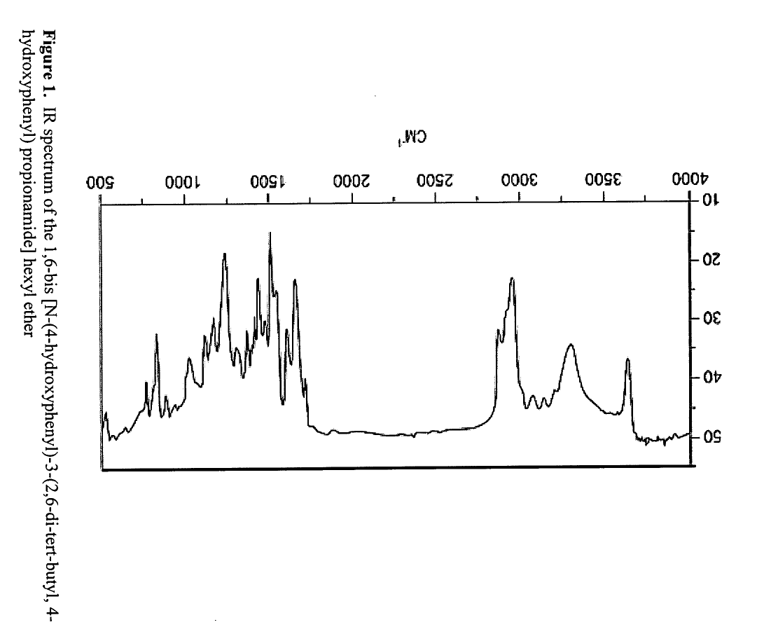

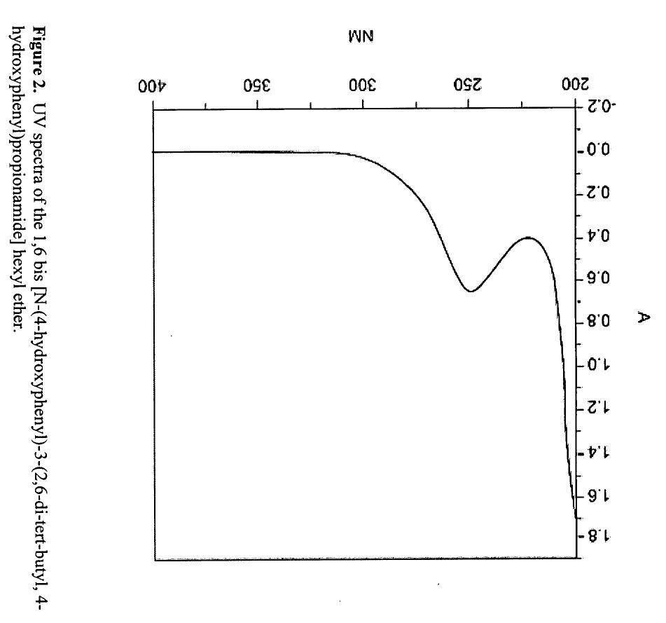

Two moles of N-(4-hydroxyphenyl)-3-(2,6-di-tert-butyl, 4-hydroxyphenyl) propionamide and one mole of 1,6-dibromo hexane were dissolved in acetone in a round bottom flask under nitrogen. To the reaction mixture was added oven dried potassium carbonate and the reaction mixture was refluxed till the completion of the reaction (monitored by HPLC/TLC). After completion, the potassium carbonate was filtered off and acetone was removed by distillation to obtain solid residue. The solid residue after washing with water gave the desired compound as a white powder with melting point 195-197° C. The product was characterized by its IR and UV spectral analysis which can be seen in FIG. 1 and FIG. 2 respectively.

### EXAMPLE 2

### Stabilization of polypropylene by 1,6-bis [N-(4-hydroxyphenyl)-3-(2,6-di-tert-butyl, 4-hydroxyphenyl)propionamide]hexyl ether

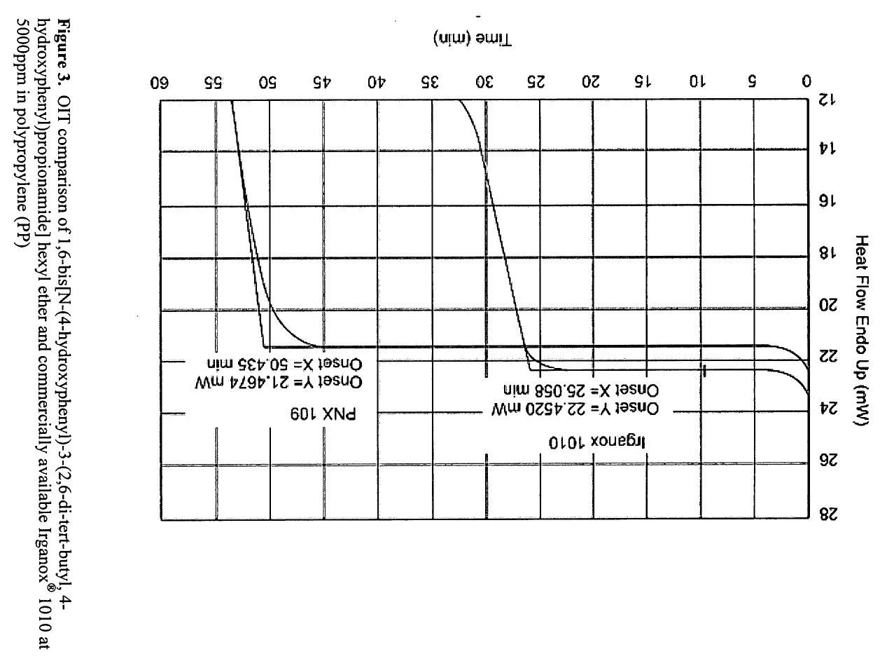

5000 ppm of 1,6-bis [N-(4-hydroxyphenyl)-3-(2,6-di-tert-butyl, 4-hydroxyphenyl)propionamide]hexyl ether was added to unstabilized polypropylene powder and extruded with single screw extruder in the form wires which was palletized. The pelltized sample of polypropylene was subjected to DSC to test for the stabilization (or Oxidative Induction Time determination). The results are shown in FIG. 3, which shows that 1,6-bis [N-(4-hydroxyphenyl)-3-(2,6-di-tert-butyl, 4-hydroxyphenyl)propionamide]hexyl ether has a significantly higher oxidative induction time than commercially available Irganox

### EXAMPLE 3

### Macromolecular Antioxidants Linked Via Linkers

3-(3,5-di-tert-butyl-4-hydroxyphenyl)-N-(4-hydroxyphenyl)propanamide was synthesized by the method described in our earlier work (Provisional Patent Application No. 60/633,196, filed Dec. 3, 2004) A linker was attached to 3-(3,5-di-tert-butyl-4-hydroxyphenyl)-N-(4-hydroxyphenyl )propanamide at the phenolic hydroxyl using methylbromoacetate. The reaction was done in dry acetone and in presence of potassium carbonate at refluxing condition.

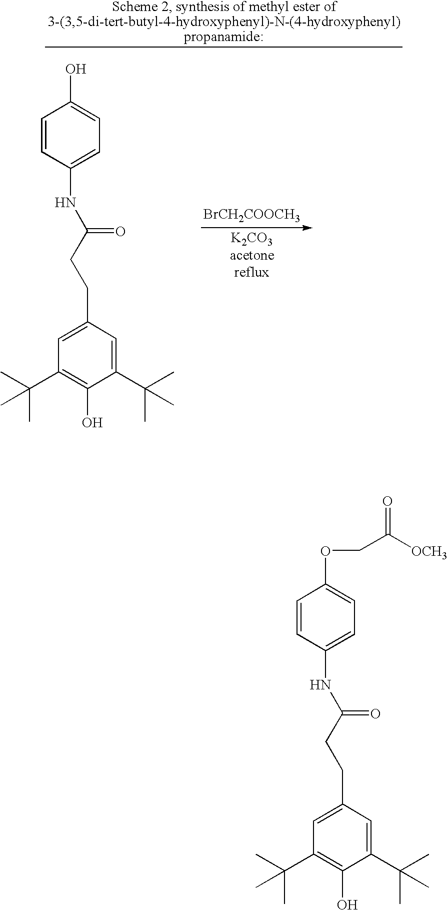

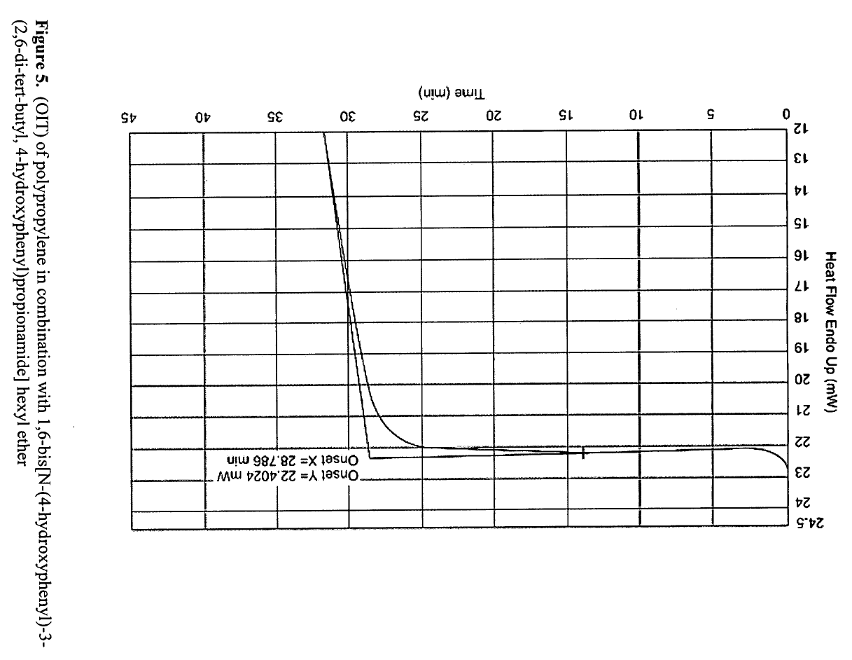

3-(3,5-di-tert-butyl-4-hydroxyphenyl)-N-(4-hydroxyphenyl)propanamide, methylbromoacetate and potassium carbonate were taken in equal molar ratio and dissolved in dry acetone. The reaction was conducted at refluxing condition and under nitrogen atmosphere. The reaction was monitored by TLC. After the consumption of 3-(3,5-di-tert-butyl-4-hydroxyphenyl)-N-(4-hydroxyphenyl)propanamide, the reaction mixture was filtered to remove the potassium carbonate and then acetone was removed on rota-vapor. Now the solid reaction mixture was dumped into ice-cooled water to get the precipitate of methyl ester of 3-(3,5-di-tert-butyl-4-hydroxyphenyl)-N-(4-hydroxyphenyl)propanamide. Methyl ester of 3-(3,5-di-tert-butyl-4-hydroxyphenyl)-N-(4-hydroxyphenyl )propanamide was characterized by NMR. Performance was checked in polypropylene at 5000 ppm using DSC which shows 28.8 min of OIT (FIG. 5).

### EXAMPLE 4

### Coupling of methyl ester of 3-(3,5-di-tert-butyl-4-hydroxyphenyl)-N-(4-hydroxyphenyl)propanamide with pentaerythritol

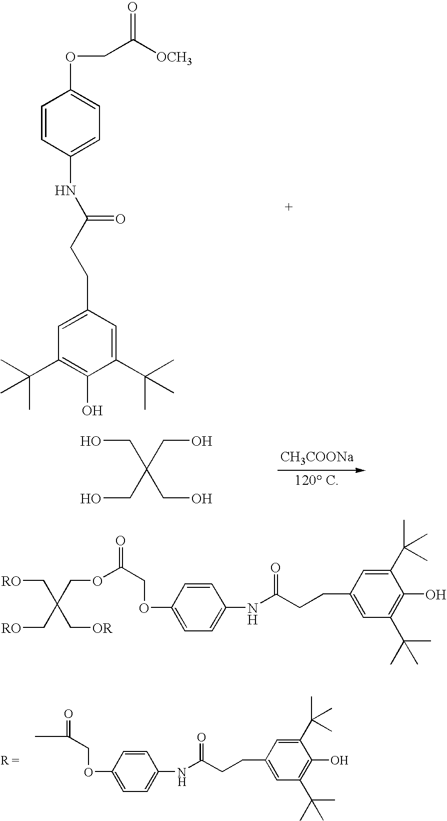

The reaction was performed in bulk. The reaction was started at 100° C. under vacuum and in nitrogen atmosphere. The temperature was raised to 120° C. after melting of the reaction mixture. The reaction was monitored by TLC. After complete conversion of pentaerythritol, the reaction was worked-up to get the pentaerythritol coupled with 13-(3,5-di-tert-butyl-4-hydroxyphenyl)-N-(4-hydroxyphenyl )propanamide and characterized by NMR.

### EXAMPLE 5

### Fe-Salen Biomimetic Catalyzed Synthesis of Polymeric Macromolecular Antioxidant N-phenyl-para-phenylene-diamine (AO-1)

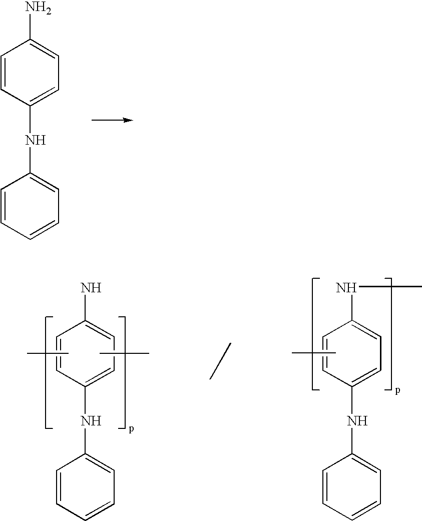

N-phenyl-p-phenylenediamine (5 g) was dissolved in THF (50 ml) and 100 mg of Fe-Salen was added to it. To the reaction mixture 25% hydrogen peroxide (equimolar) solution was added incrementally over the period of 1 hour. After completion of addition, the reaction mixture was stirred for additional 24 hours. After completion of reaction THF was removed, product washed with water and dried

### EXAMPLE 6

### Fe-Salen Biomimetic Catalyzed Synthesis of Polymeric Macromolecular Antioxidant Diaminonapthlene (AO-2).

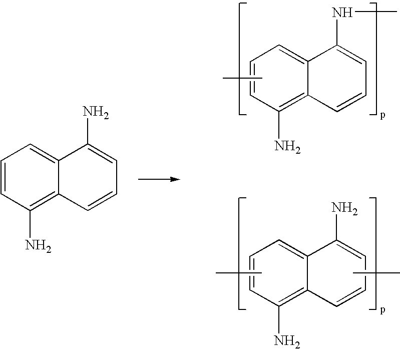

1,5-diamino-napthalene (5 g) was dissolved in THF (50 ml) and 100 mg of Fe-Salen was added to it. To the reaction mixture 25% hydrogen peroxide (equimolar) solution was added incrementally over the period of 1 hour. After completion of addition, the reaction mixture was stirred for additional 24 hours. After completion of reaction THF was removed, product washed with water and dried.

### EXAMPLE 7

### HRP Catalyzed Synthesis of Copolymeric Macromolecular Antioxidant n-phenyl-para-phenylene-diamine and Napthylamine (AO-3).

N-phenyl-p-phenylenediamine (3 g) and 1-amino-napthalene (2 g) were dissolved in MeOH: pH═4.3 (100 ml) phosphate buffer and 100 mg of HRP enzyme was added to it. To the reaction mixture 5% hydrogen peroxide (equimolar) solution was added incrementally over the period of 3 hours. After completion of addition, the reaction mixture was stirred for additional 24 hours. After completion of reaction methanol and water were removed, and the product was washed with water and dried.

### EXAMPLE 8

### Evaluation of Polymeric Macromolecular Antioxidants in Synthetic Ester Based Lubricant Oil

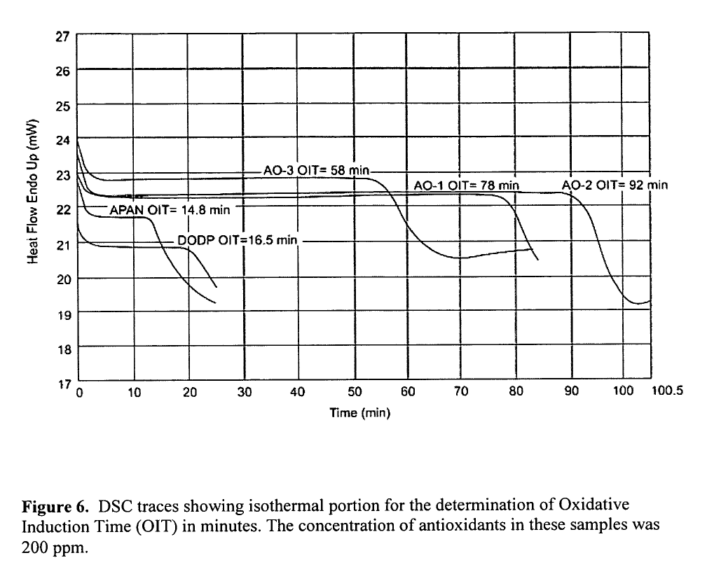

The oxidative stability of the polyol ester base stock samples containing 200 ppm by weight of polymeric macromolecular antioxidants were evaluated on the basis of their OIT values. FIG. 6 shows the isothermal DSC curves representing the exothermic thermal-oxidative degradation at 200° C. for polyol ester base stock. Data in FIG. 6 suggests that the sample containing commercially used APAN (alkylated phenyl naphthalene amine) and DODP (di-octylated diphenyl amine) have significantly lower resistance to oxidative degradation compared to polymeric macromolecular antioxidants. The OIT values for the samples containing 200 ppm of commercial antioxidants are 14.8 min and 16.5 min, respectively. On the other hand, the samples containing 200 ppm polymeric macromolecular antioxidants AO1, AO2 and AO3 showed significantly higher OIT values of 78 min, 92 min and 58 min, respectively.

### EXAMPLE 9

### Evaluation of Polymeric Macromolecular Antioxidants in Polyolefins

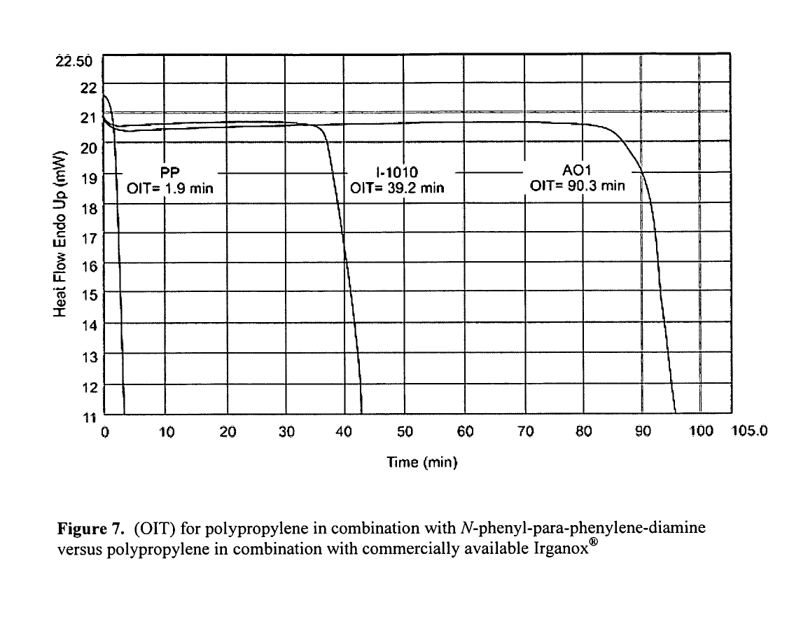

The isothermal oxidative induction time (OIT) is used to compare the performance macromolecular antioxidant in polyolefins. The polypropylene (PP) samples were extruded into small pellets by mixing with 5000 ppm by weight of antioxidants. The OIT values for PP containing macromolecular antioxidant AO1 and Irganox® 1010 are 90 minutes and 39 minutes, respectively (FIG. 7)

What is claimed is:

1. A compound represented by a structural formula selected from I-VI 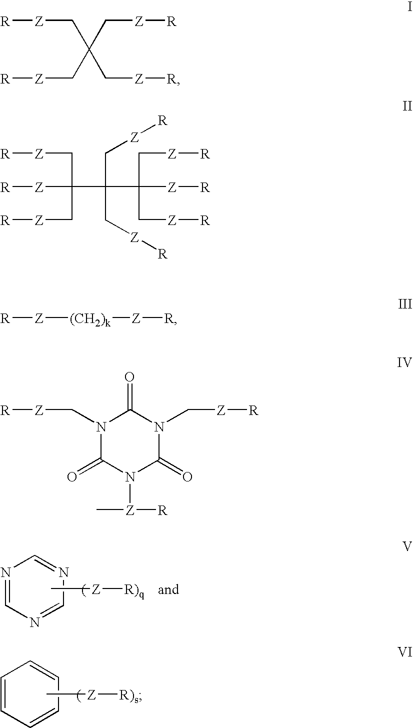
wherein:
Z, for each occurrence, is independently a bond, an optionally substituted alkylene group, —S—, —O— or —NH—;
R is: 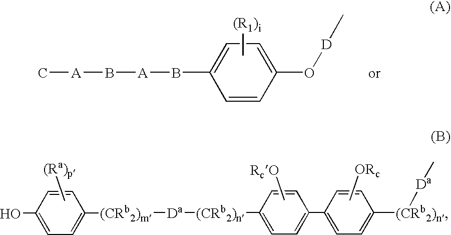
wherein:
A in each occurrence, independently is a bond, —O—, —NH—, —S—, —C(O)—, —C(O)NH—, —NHC(O)—, —C(O)O—, —OC(O)—, —CH═N— or —N═CH—;
B in each occurrence, independently is a bond or an optionally substituted alkylene group;
C in each occurrence independently is —H, an optionally substituted alkylene group or 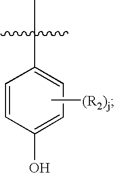
R₁ and R₂ in each occurrence, independently is an optionally substituted alkyl, optionally substituted aryl or optionally substituted aralkyl;
R_{c} and R_{c}′ are independently H or an optionally substituted alkyl;
R^{a}, for each occurrence, is independently an optionally substituted alkyl, 20 optionally substituted aryl, optionally substituted alkoxycarbonyl, optionally substituted ester, —OH, —NH₂, —SH;
R^{b}, for each occurrence, is independently H or optionally substituted alkyl;
p′, for each occurrence, is independently integers from 0 to 4;
m′ and n′, for each occurrence, are independently integers from 0 to 6;
D in each occurrence, independently is a bond, an optionally substituted alkylene group, —(CH₂)₁C(O)O(CH₂)ₕ—, —(CH₂)₁NHC(O)(CH₂)ₕ—, —(CH₂)₁C(O)NH(CH₂)ₕ—, —(CH₂)₁C(O)O(CH₂)ₕ—, —(CH₂)₁OC(O)(CH₂)ₕ—, —(CH₂)₁CH═N(CH₂)ₕ—, —(CH₂)₁N═CH(CH₂)ₕ—, —(CH₂)₁NH(CH₂)ₕ—, —(CH₂)₁S—(CH₂)ₕ—, —(CH₂)₁O(CH₂)ₕ— or —(CH₂)₁C(O)(CH₂)ₕ—;
D^{a}, for each occurrence, is independently —C(O)NR_{d}—, —NR_{d}C(O)—, —NR_{d}—, —CR_{d}═N—, —C(O)—, —C(O)O—, —OC(O)—, —O—, —S—, —C(O)OC(O)— or a bond, wherein R_{d} is independently H or optionally substituted alkyl;
i and j, for each occurrence, are independently 0, 1, 2, 3 or 4;
k is a positive integer from 1 to 12;
l is 0 or a positive integer from 1 to 12;
h is 0 or a positive integer from 1 to 12;
s is a positive integer from 1 to 6; and
q is a positive integer from 1 to 3.

2. The compound of claim 1, wherein the compound is represented by structural formula I.

3. The compound of claim 2, wherein:
R is: 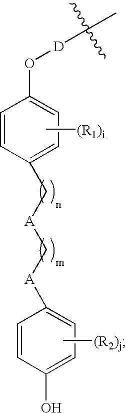
wherein:
n and m in each occurrence, independently is an integer from 0 to 12.

4. The compound of claim 3, wherein:
R₁ and R₂ in each occurrence, independently is —H or optionally substituted alkyl; and
i and j in each occurrence, independently is 0, 1 or 2.

5. The compound of claim 4, wherein
R is: 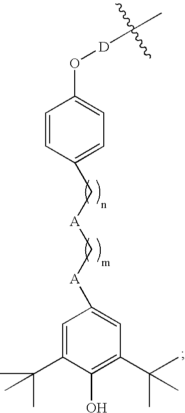
wherein:
n and m in each occurrence, independently is an integer from 0 to 6.

6. The compound of claim 5, wherein:
A in each occurrence, independently is a bond, —C(O)NH— or —CH₂—.

7. The compound of claim 5, wherein:
A in each occurrence, independently is a bond, —NHC(O)—, or —CH₂—.

8. The compound of claim 7, wherein
R is: 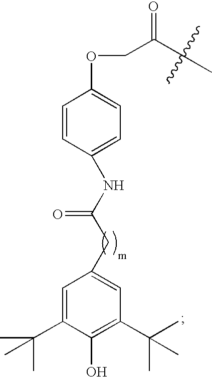
wherein m is an integer from 0 to 6.

9. The compound of claim 8, wherein m is 2.

10. The compound of claim 2, wherein R is represented by Structural Formula B:

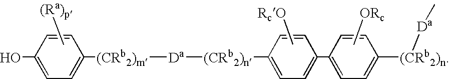

11. The compound of claim 10, wherein:
D^{a}, for each occurence, is independently —C(O)O—, —OC(O)—, —C(O)NH—, —NHC(O)—, —NH—, —O— or —C(O)—;
R_{c} and R_{c}′ are independently H or optionally substituted alkyl and at least one R_{c} and R_{c}′ is H;
R^{b} is H;
R^{a}, for each occurrence is independently an optionally substituted alkyl or optionally substituted alkoxycarbonyl;
n′ and m′, for each occurrence, are independently integers from 0 to 2;
and
p′, for each occurrence, is independently an integer from 0 to 2.

12. The compound of claim 11, wherein:
D^{a} is —NH—, —C(O)NH— or —NHC(O)—;
R^{a}, for each occurrence is independently an alkyl or an alkoxycarbonyl; and
p′ is 2.

13. The compound of claim 12, wherein each R^{a} is independently an alkyl group.

14. The compound of claim 10, wherein R is represented by Structural Formula B1: 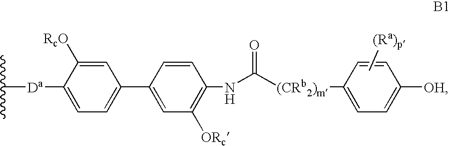
wherein:
R_{c} and R_{c}′ are independently H or optionally substituted alkyl and at least one of R_{c} and R_{c}′ is H;
Rb is H;
R^{a}, for each occurrence is independently an optionally substituted alkyl or optionally substituted alkoxycarbonyl;
n′ and m′, for each occurrence, are independently integers from 0 to 2;
p′, for each occurrence, is independently an integer from 0 to 2; and
D^{a} is —NH—, —C(O)NH— or —NHC(O)—.

15. The compound of claim 14, wherein:
R^{a}, for each occurrence, is independently an optionally substituted alkyl;
R^{b} is H;
p′, for each occurrence, is independently an integer from 0 to 2; and
m′, for each occurrence, is independently an integer from 0 to 2.

16. The compound of claim 15, wherein R^{a} is independently an alkyl and p′ is 2.

17. The compound of claim 10, wherein R is represented by Structural Formula B2: 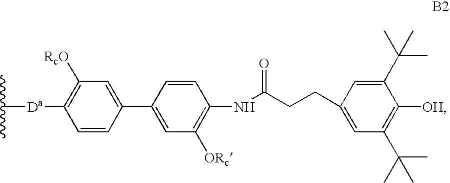
wherein R_{c} and R_{c}′ are independently H or optionally substituted alkyl and at least one of R_{c} and R_{c}′ is H; and D^{a} is —NH—, —C(O)NH— or —NHC(O)—.

18. The compound of claim 17, wherein R_{c} and R_{c}′ are H.

19. The compound of claim 17, wherein R_{c} is H and R_{c}′ is an alkyl.

20. The compound of claim 19, wherein R_{c}′ is an C1-C10 alkyl.

21. The compound of claim 20, wherein R_{c}′ is an C10 alkyl.

22. The compound of claim 20, wherein R_{c}′═—(CH₂)₉CH₃.

23. The compound of claim 17, wherein R_{c}′ is H and R_{c} is an alkyl.

24. The compound of claim 23, wherein R_{c} is an C1-C10 alkyl.

25. The compound of claim 24, wherein R_{c} is an C10 alkyl.

26. The compound of claim 25, wherein R_{c}′═—(CH₂)₉CH₃.

27. The compound of claim 1, wherein the compound is represented by structural formula III.

28. The compound of claim 27, wherein:
R is: 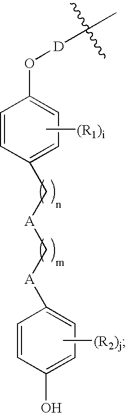
wherein:
n and m in each occurrence, independently is an integer from 0 to 12.

29. The compound of claim 28, wherein:
R₁ and R₂ in each occurrence, independently is —H or optionally substituted alkyl; and
i and j in each occurrence, independently is 0, 1 or 2.

30. The compound of claim 29, wherein
R is: 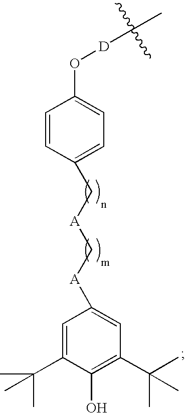
wherein:
n and m in each occurrence, independently is an integer from 0 to 6.

31. The compound of claim 30, wherein:
A in each occurrence, independently is a bond, —C(O)NH— or —CH₂—.

32. The compound of claim 30, wherein:
A in each occurrence,-independently is a bond, —NHC(O)—, or —CH₂—.

33. The compound of claim 32, wherein
R is: 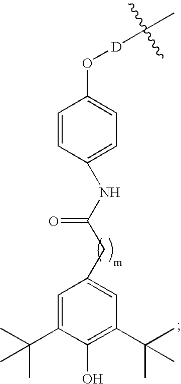
wherein m is an integer from 0 to 6.

34. The compound of claim 33, wherein m is 2.

35. The compound of claim 27, wherein R is represented by Structural Formula B:

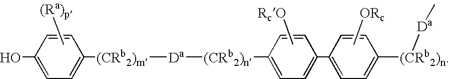

36. The compound of claim 35, wherein:
D^{a}, for each occurrence, is independently —C(O)O—, —OC(O)—, —C(O)NH—, —NHC(O)—, —NH—, —O— or —C(O)—;
R_{c} and R_{c}′ are independently H or optionally substituted alkyl and at least one R_{c} and R_{c}′ is H;
R^{b} is H;
R^{a}, for each occurrence is independently an optionally substituted alkyl or optionally substituted alkoxycarbonyl;
n′ and m′, for each occurrence, are independently integers from 0 to 2; and
p′, for each occurrence, is independently an integer from 0 to 2.

37. The compound of claim 36, wherein:
D^{a} is —NH—, —C(O)NH— or —NHC(O)—;
R^{a}, for each occurrence is independently an alkyl or an alkoxycarbonyl; and
p′ is 2.

38. The compound of claim 37, wherein each R^{a} is independently an alkyl group.

39. The compound of claim 35, wherein R is represented by Structural Formula B1: 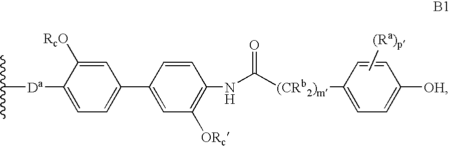
wherein:
R_{c} and R_{c}′ are independently H or optionally substituted alkyl and at least one of R_{c} and R_{c}′ is H;
R^{b} is H;
R^{a}, for each occurrence is independently an optionally substituted alkyl or optionally substituted alkoxycarbonyl;
n′ and m′, for each occurrence, are independently integers from 0 to 2;
p′, for each occurrence, is independently an integer from 0 to 2; and
D^{a} is —NH— or —C(O)NH—.

40. The compound of claim 39, wherein:
R^{a}, for each occurrence, is independently an optionally substituted alkyl;
R^{b} is H;
p′, for each occurrence, is independently an integer from 0 to 2; and
m′, for each occurrence, is independently an integer from 0 to 2.

41. The compound of claim 40, wherein R^{a} is independently an alkyl and p′ is 2.

42. The compound of claim 35, wherein R is represented by Structural Formula B2: 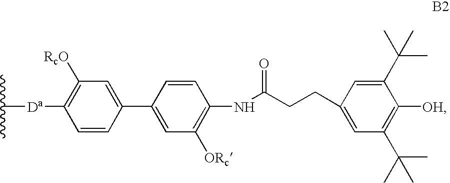
wherein R_{c} and R_{c}′ are independently H or optionally substituted alkyl and at least one of R_{c} and R_{c}′ is H; and D^{a} is —NH— or —C(O)NH—.

43. The compound of claim 42, wherein R_{c} and R_{c}′ are H.

44. The compound of claim 42, wherein R_{c} is H and R_{c}′ is an alkyl.

45. The compound of claim 44, wherein R_{c}′ is an C1-C10 alkyl.

46. The compound of claim 45, wherein R_{c}′ is an C10 alkyl.

47. The compound of claim 46, wherein R_{c}′═—(CH₂)₉CH₃.

48. The compound of claim 42, wherein R_{c}′ is H and R_{c} is an alkyl.

49. The compound of claim 48, wherein R_{c} is an C1-C10 alkyl.

50. The compound of claim 49, wherein R_{c} is an C10 alkyl.

51. The compound of claim 50, wherein R_{c}′═—(CH₂)₉CH₃.

52. A polymer comprising at least one repeating unit selected from Structural Formulas VIIa, VIIb, VIIIa, VIIIb or a combination thereof: 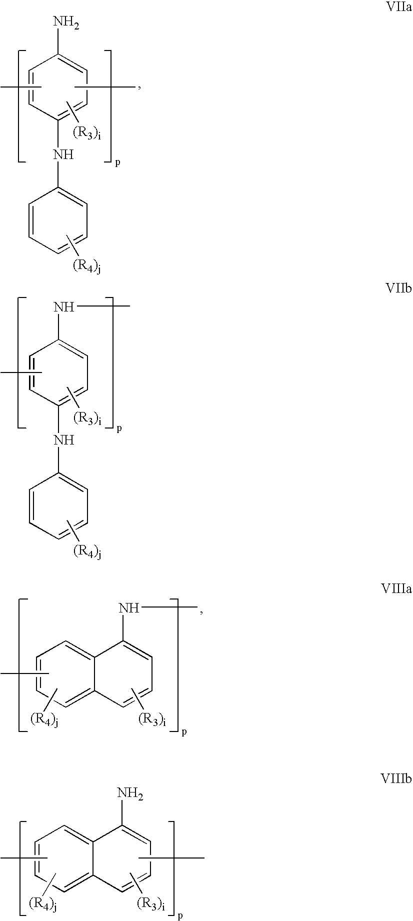
or a combination thereof, wherein:
R₃ and R4 in each occurrence, independently is C1-C16 alkyl, —O—C1-C16 alkyl, —NHAr, —NH₂, —OH, or —SH;
i and j in each occurrence, independently is 0, 1, 2, 3 or 4; and
p in each occurrence, independently is an integer equal to or greater than 2.

53. The polymer of claim 52, wherein the polymer comprises at least one repeating unit represented by a structural formula selected from VIIa, VIIb or a combination thereof.

54. The polymer of claim 53, wherein:
i and j are 0.

55. The polymer of claim 52, wherein the polymer comprises at least one repeating unit represented by a structural formula selected from VIIIa, VIIIb or a combination thereof.

56. The polymer of claim 55, wherein:
i is 0; and
j is 1.

57. The polymer of claim 52, wherein the polymer comprises at least one repeating unit represented by a structural formula selected from: 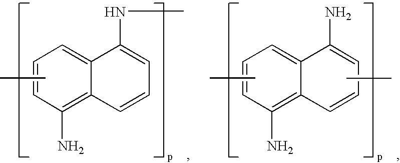
or a combination thereof.

58. A method of preventing oxidation comprising combining an oxidizable material with a compound represented by a structural formula selected from I-VI: 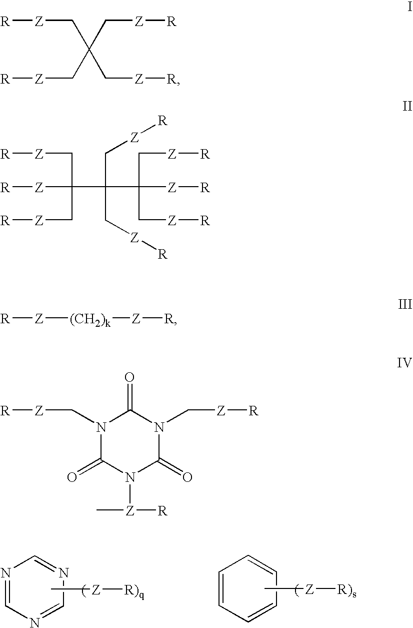
wherein:
Z, for each occurrence, is independently a bond, an optionally substituted alkylene group, —S—, —O— or —NH—,
R is: 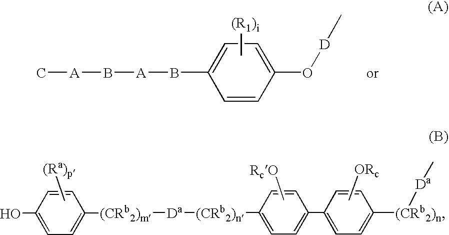
wherein:
A in each occurrence, independently is a bond, —O—, —NH—, —S—, —C(O)—, —C(O)NH—, —NHC(O)—, —C(O)O—, —OC(O)—, —CH═N— or —N═CH—;
B in each occurrence, independently is a bond or an optionally substituted alkylene group;
C in each occurrence independently is —H, an optionally substituted alkylene group or 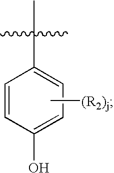
R₁ and R₂ in each occurrence, independently. is an optionally substituted alkyl, optionally substituted aryl or optionally substituted aralkyl;
R_{c} and R_{c}′ are independently H or an optionally substituted alkyl;
R^{a}, for each occurrence, is independently an optionally substituted alkyl, optionally substituted aryl, optionally substituted alkoxycarbonyl, optionally substituted ester, —OH, —NH₂, —SH;
R^{b}, for each occurrence, is independently H or optionally substituted alkyl;
p′, for each occurrence, is independently integers from 0 to 4;
m′ and n′, for each occurrence, are independently integers from 0 to 6;
D in each occurrence, independently is a bond, an optionally substituted alkylene group, —(CH₂)₁C(O)O(CH₂)ₕ—, —(CH₂)₁ NHC(O)(CH₂)ₕ—, —(CH₂)₁C(O)NH(CH₂)ₕ—, —(CH₂)₁C(O)O(CH₂)ₕ—, —(CH₂)₁OC(O)(CH₂)ₕ—, —(CH₂)₁CH═N(CH₂)ₕ—, —(CH₂)₁N═CH(CH₂)ₕ—, —(CH₂)₁NH(CH₂)ₕ—, —(CH₂)₁S—(CH₂)ₕ—, —(CH₂)₁O(CH₂)ₕ— or —(CH₂)₁C(O)(CH₂)ₕ—;
D^{a}, for each occurrence, is independently —C(O)NR_{d}—, —NR_{d}C(O)—, —NR_{d}—, —CR_{d}—═N—, —C(O)—, —C(O)O—, —OC(O)—, —O—, —S—, —C(O)OC(O)— or a bond, wherein R_{d} is independently H or optionally substituted alkyl;
i and j, for each occurrence, are independently 0, 1, 2, 3 or 4;
k is a positive integer from 1 to 12;
l is 0 or a positive integer from 1 to 12;
h is 0 or a positive integer from 1 to 12
s is a positive integer from 1 to 6; and
q is a positive integer from 1 to 3.

59. A method of preventing oxidation comprising combining an oxidizable material with a polymer comprises at least one repeating unit represented by a structural formula selected from VIIa, VIIb, VIIIa ,VIIIb or a combination thereof: 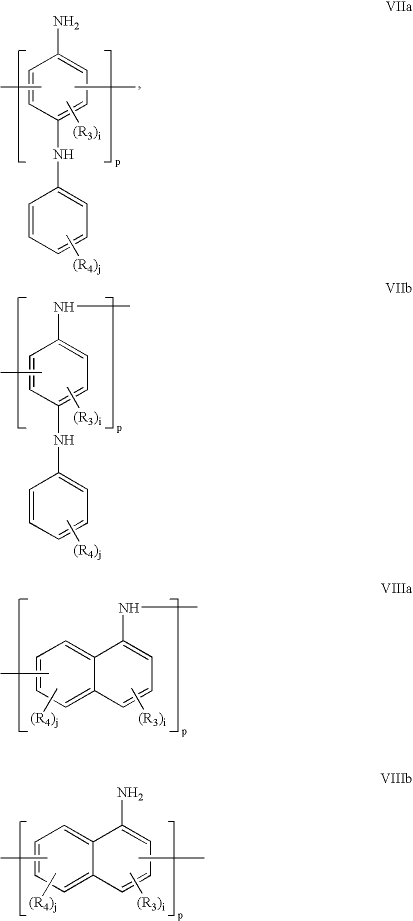
R₃ and R₄ in each occurrence, independently is C1-C16 alkyl, —O—C1-C16 alkyl, —NHAr, —NH₂, —OH, or —SH;
i and j in each occurrence, independently is 0, 1, 2, 3 or 4; and
p in each occurrence, independently is an integer equal to or greater than 2.

60. A method of synthesizing a macromolecular antioxidant represented by a structural formula selected from I-VI: 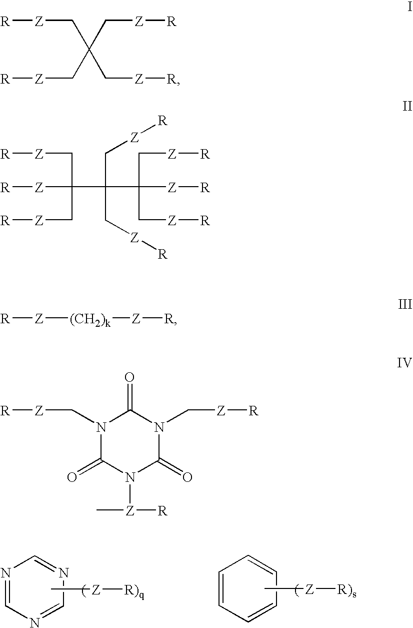
wherein:
Z, for each occurrence, is independently a bond, an optionally substituted
alkylene group, —S—, —O— or —NH—;
R is 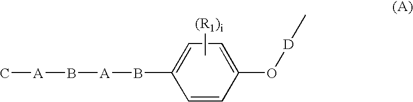
wherein:
A in each occurrence, independently is a bond, —O—, —NH—, —S—, —C(O)—, —C(O)NH—, —NHC(O)—, —C(O)O—, —OC(O)—, —CH═N— or —N═CH—;
B in each occurrence, independently is a bond or an optionally substituted alkylene group;
C in each occurrence independently is —H, an optionally substituted alkylene group or 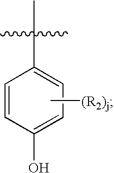
R₁ and R₂ in each occurrence, independently is an optionally substituted alkyl, optionally substituted aryl or optionally substituted aralkyl;
D in each occurrence, independently is a bond, an optionally substituted alkylene group, —(CH₂)₁C(O)O(CH₂)ₕ—, —(CH₂)₁NHC(O)(CH₂)ₕ—, —(CH₂)₁C(O)NH(CH₂)ₕ—, —(CH₂)₁C(O)O(CH₂)ₕ—, —(CH₂)₁OC(O)(CH₂)ₕ—, —(CH₂)₁CH═N(CH₂)ₕ—, —(CH₂)₁N═CH(CH₂)ₕ—, —(CH₂)₁NH(CH₂)ₕ—, —(CH₂)₁S—(CH₂)ₕ—, —(CH₂)₁O(CH₂)ₕ— or —(CH₂)₁C(O)(CH₂)ₕ—;
i and j, for each occurrence, are independently 0, 1, 2, 3 or 4;
k is a positive integer from 1 to 12;
l is 0 or a positive integer from 1 to 12;
h is 0 or a positive integer from 1 to 12;
s is a positive integer from 1 to 6; and
q is a positive integer from 1 to 3.
comprising the step of reacting R⁺⁺, wherein R⁺⁺ is 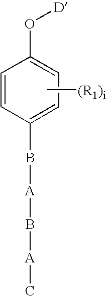
with a compound selected from: 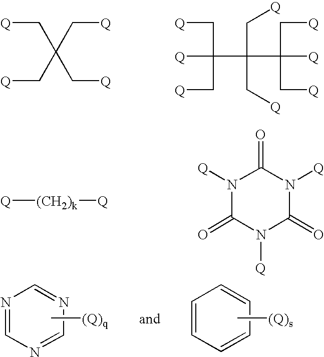
Q is a halogen or -Z-H;
D′ in each occurrence, independently is —H, an optionally substituted alkyl group, —(CH₂)₁C(O)O(CH₂),R*—, —(CH₂)₁NHC(O)(CH₂)ₕR*—, —(CH₂)₁C(O)NH(CH₂)ₕR*—, —(CH₂)₁C(O)O(CH₂)ₕR*—, —(CH₂)₁OC(O)(CH₂)ₕR*—, —(CH₂)₁CH═N(CH₂)ₕR*—, —(CH₂)₁N═CH(CH₂)ₕR*—, —(CH₂)₁NH(CH₂)ₕR*—, —(CH₂)₁S—(CH₂)ₕR*—, —(CH₂)₁O(CH₂)ₕR*— or —(CH₂)₁C(O)(CH₂)ₕR*—; and
R* in each occurrence, independently is —CH₃ or —H.

61. The method of claim 60, wherein
R ⁺⁺ is:

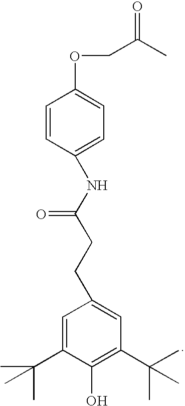

62. The method of claim 60, wherein R⁺⁺ is reacted with the compound in the presence of the sodium acetate .

63. The method of claim 62, wherein R⁺⁺ and the compound are reacted under nitrogen.

64. The method of claim 63, wherein R⁺⁺ and the compound are reacted at a temperature of from about 150° C. to about 50° C.

65. A method of synthesizing a macromolecular antioxidant represented by a structural formula selected from I-VI 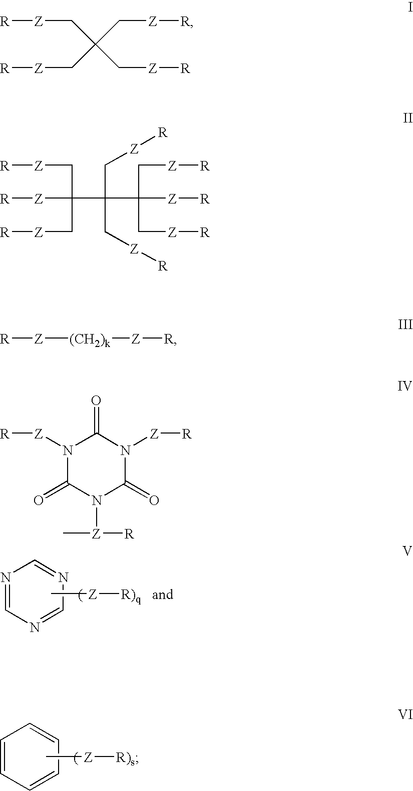
wherein:
Z, for each occurrence, is independently a bond, an optionally substituted
alkylene group, —S—, —O— or —NH—;
R is: 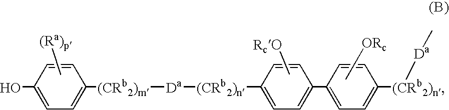
wherein:
R_{c} and R_{c}′ are independently H or an optionally substituted alkyl;
R^{a}, for each occurrence, is independently an optionally substituted alkyl, optionally substituted aryl, optionally substituted alkoxycarbonyl, optionally substituted ester, —OH, —NH₂, —SH;
R^{b}, for each occurrence, is independently H or optionally substituted alkyl;
p′, for each occurrence, is independently integers from 0 to 4;
m′ and n′, for each occurrence, are independently integers from 0 to 6;
D^{a}, for each occurrence, is independently —C(O)NR_{d}—, —NR_{d}C(O)—, —NR_{d}—, —CR_{d}═N—, —C(O)—, —C(O)O—, —OC(O)—, —O—, —S—, —C(O)OC(O)— or a bond, wherein R_{d} is independently H or optionally substituted alkyl;
k is a positive integer from 1 to 12;
s is a positive integer from 1 to 6; and
q is a positive integer from 1 to 3,
comprising the step of reacting R₂⁺⁺, wherein R₂⁺⁺ is: 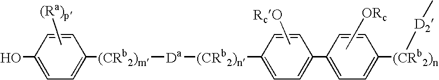
with a compound selected from: 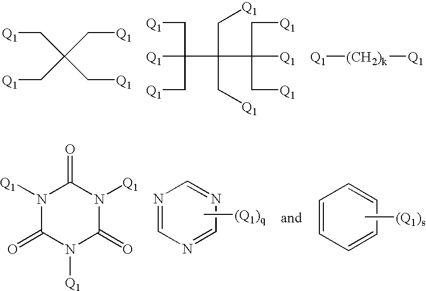
wherein:
D₂′ is D₂ₐ′ or D_{2b}′;
Q₁ is Q₁ₐ or Q_{1b}; wherein when D₂′ is D₂ₐ′, Q₁ is Q₁ₐ and when D₂′ is D_{2b}′, Q₁ is Q_{1b};
D₂ₐ′ is —C(O)—X and X is H or a leaving group;
D_{2b}′ is NHR_{d}, —SH, or —OH, wherein R_{d} is H or optionally substituted alkyl;
Q₁ₐ is a nucleophile;
Q_{1b} is a —W—X₁, wherein X₁ is a leaving group and W is a bond or —C(O)—.

66. A method of synthesizing a polymer comprising at least one repeating unit selected from structural formulas VIIa, VIIb, VIIIa, VIIIb or a combination thereof: 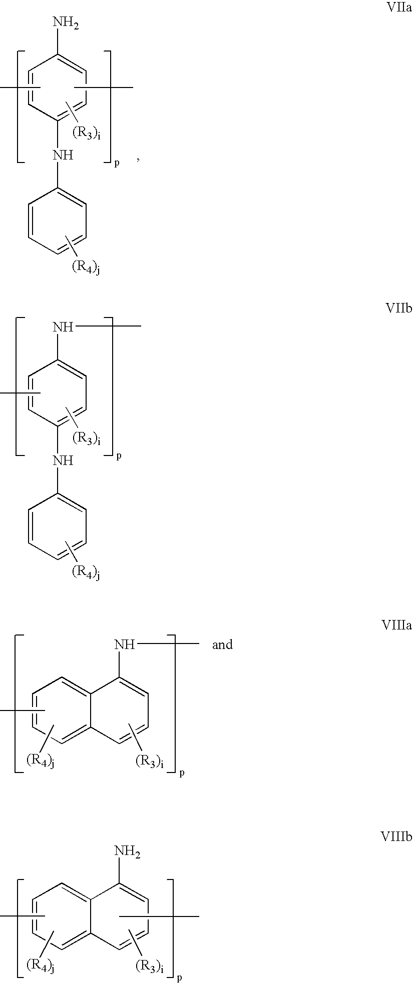
R₃ and R₄ in each occurrence, independently is C1-C16 alkyl, —O—C1-C16 alkyl, —NHAr, —NH₂, —OH, or —SH;
i and j in each occurrence, independently is 0, 1, 2, 3 or 4; and
p in each occurrence, independently is an integer equal to or greater than 2; comprising the steps of polymerizing a monomer represented by a structural formula selected from: 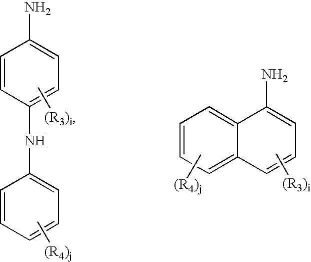
or combinations thereof in the presence of an oxidative polymerization catalyst.

67. The method of claim 66, wherein the oxidative polymerization catalyst is a biocatalyst or a biomimetic catalyst selected from Iron(II)-salen complexes, horseradish peroxidase, soybean peroxidase, hematin, laccase, tyroniase, ferric chloride, ammonium persulphate and a tyroniase-model complex.

68. The method of claim 67, wherein the oxidative polymerization catalyst is an inorganic or organometallic catalyst
